(19) United States
(12) Patent Application Publication
(10) Pub. No.: US 2010/0324543 A1
(43) Pub. Date: Dec. 23, 2010

(54) Method And Apparatus For Integrating Cataract Surgery With Glaucoma Or Astigmatism Surgery

(76) Inventors: Ronald M. Kurtz, Irvine, CA (US); Tibor Juhasz, Corona del Mar, CA (US)

Correspondence Address: FISH & RICHARDSON P.C. (SD), P.O. BOX 1022, MINNEAPOLIS, MN, 55440-1022, US

(21) Appl. No.: 12/823,072

(22) Filed: Jun. 24, 2010

Related U.S. Application Data
(63) Continuation-in-part of application No. 12/233,401, filed on Sep. 18, 2008.
(60) Provisional application No. 60/973,405, filed on Sep. 18, 2007.

(51) Int. Cl. A61F 9/008

(52) U.S. Cl. 606/6

(57) ABSTRACT

A method for integrated eye surgery can include determining a cataract-target region in a lens of the eye; applying cataract-laser pulses to photodisrupt a portion of the determined cataract-target region; determining a glaucoma-target region or an astigmatism-target region in a peripheral region of the eye; and applying surgical laser pulses to create one or more incisions in the glaucoma- or astigmatism-target region by photodisruption; wherein the steps of the method are performed within an integrated surgical procedure. The laser pulses can be applied before making an incision on a cornea of the eye. The integrated surgical procedure may involve using the same pulsed laser source for three functions: for photodisrupting the target region, for making an incision on the capsule of the lens and for making an incision on the cornea of the eye.

### CROSS-REFERENCE TO RELATED APPLICATION

This application is a continuation-in-part of and claims the benefit of the priority of U.S. patent application Ser. No. 12/233,401, filed Sep. 18, 2008, and entitled “Methods and Apparatus for Integrated Cataract Surgery”, which claims the benefit of the priority of Provisional Application Ser. No. 60/973,405, entitled, “Methods and Apparatus for Integrated Cataract Surgery”; both prior applications are hereby incorporated by reference.

### BACKGROUND

This patent document relates to techniques, apparatus and systems for integrating cataract surgery with glaucoma or astigmatism surgeries.

Cataract surgery is one of the most common ophthalmic procedures performed. The primary goal of cataract surgery is the removal of the defective lens and replacement with an artificial lens or intraocular lens (IOL) that restores some of the optical properties of the defective lens. Generally, the IOL is capable of improving the transmission of light, and reduce the scattering, the absorption or both.

A widely practiced form of cataract surgery involves ultrasound-based phacoemulsification. During this type of surgery the lens of the eye is entered through an incision with a phaco probe. The probe generates ultrasound which breaks up the lens into small fractions, leading to its emulsification. Remarkably, this procedure has remained largely unchanged over the past twenty years. In the course of cataract surgery based on phaco-emulsification, a series of individual surgical maneuvers are undertaken, including (1) Corneal incision and paracentesis; (2) Injection of a viscoelastic to maintain the overall structure anterior chamber and to prevent its collapse; (3) Incision of anterior capsule; (4) Creation of anterior capsulorhexis; (5) Hydrodissection of lens nucleus; (6) Fragmentation of the lens nucleus by mechanical and ultrasound-based methods (7) Aspiration of lens nucleus; (8) Injection of viscoelastic into capsular bag; (9) Aspiration of lens cortical material; (10) Insertion and positioning of intraocular lens; (11) Removal of viscoelastic; and (12) Examination of corneal wound integrity, possible suture placement. Some of these steps are necessitated by the fact that the eye is opened up during the eye surgery and entered physically with instruments to break up and remove the lens.

Cataract surgery performed in this manner involves a high level of skill by the surgeon and specialized equipment and supplies, many of which require the assistance of a scrub nurse. Because each step is separate from the others, it may be difficult to optimally coordinate the steps with one another during the procedure.

### SUMMARY

Briefly and generally, implementation of the present invention include a method for integrated eye surgery, including the steps of: determining a cataract-target region in a lens of the eye; applying cataract-laser pulses to photodisrupt a portion of the determined cataract-target region; determining a glaucoma-target region in a peripheral region of the eye; and applying glaucoma-laser pulses to create one or more incisions in the glaucoma-target region by photodisruption; wherein the steps of the method are performed within an integrated surgical procedure.

In some implementations, the applying the cataract-laser pulses step is performed before the applying the glaucoma-laser pulses step.

In some implementations, the applying the cataract-laser pulses step is performed after the applying the glaucoma-laser pulses step.

In some implementations, the applying the cataract-laser pulses step is performed at least partially concurrently with the applying the glaucoma-laser pulses step.

In some implementations, the applying glaucoma-laser pulses step can include applying laser pulses into at least one of a sclera, a limbal region, an ocular angle portion, or an iris root.

In some implementations, the applying glaucoma-laser pulses step can include applying laser pulses according to a pattern in relation to at least one of a trabeculoplasty, iridotomy or iridectomy.

In some implementations, the applying glaucoma-laser pulses step can include applying laser pulses to form at least one of a drain channel and a humor outflow opening.

In some implementations, the method includes inserting an implantable device into one of the drain channel or the humor outflow opening.

In some implementations, the drain channel and a humor outflow opening is configured to connect an anterior chamber of a surgical eye to a surface of the surgical eye, thereby allowing a reduction of an intraocular pressure of an aqueous humor in the surgical eye.

Some implementations may include utilizing one laser for applying both the cataract-laser pulses and the glaucoma-laser pulses.

In some implementations, the applying glaucoma-laser pulses step comprising: applying the glaucoma-laser pulses to an optimized glaucoma-target region, wherein a location of the optimized glaucoma-target region is selected to scatter the glaucoma-laser pulses less than a sclera of the eye, and to perturb an optical pathway of the eye by the formed drain channel less than a centrally formed drain channel.

In some implementations the glaucoma-target region is one of a limbus-sclera boundary region or a limbus-cornea intersection region.

In some implementations, the applying glaucoma-laser pulses step comprising: applying the glaucoma-laser pulses to form a drain channel in a direction selected to optimize the competing requirements of scattering the glaucoma-laser pulses less than a sclera of the eye, and perturbing an optical pathway eye less than a centrally formed drain channel.

In some implementations, determining a placement of the cataract-laser pulses and a placement of the glaucoma-laser pulses can be performed in a coordinated manner.

In some implementations, the method can include imaging a photodisruption achieved by the cataract-laser pulses; and determining at least portions of the glaucoma-target region in response to the imaged photodisruption.

In some implementations, the method can include imaging a photodisruption by the glaucoma-laser pulses; and determining at least portions of the cataract-target region in response to the imaged photodisruption.

In some implementations, the cataract-laser pulses are applied with a cataract-laser wavelength λ-c; and the glaucoma-laser pulses are applied with a glaucoma-laser wavelength λ-g.

In some implementations, the cataract-laser pulses are applied through a cataract-patient interface; and the glaucoma-laser pulses are applied through a glaucoma-patient interface.

In some implementations, a multi-purpose ophthalmic surgical system may include a multi-purpose laser, configured to place cataract-laser pulses into a cataract-target region, and to place glaucoma-laser pulses into a glaucoma-target region; and an imaging system, configured to image a photodisruption caused by at least one of the cataract-laser pulses and the glaucoma-laser pulses.

In some implementations, the multi-purpose ophthalmic surgical system ma is configured to apply the cataract-laser pulses at a cataract-laser wavelength λ-c, and to apply the glaucoma-laser pulses at a glaucoma-laser wavelength of λ-g.

In some implementations, the multi-purpose laser is configured to apply the cataract-laser pulses through a cataract-patient interface, and to apply the glaucoma-laser pulses through a glaucoma-patient interface.

In some implementations, the multi-purpose ophthalmic surgical system is configured to apply the cataract-laser pulses and the glaucoma-laser pulses by the same laser.

In some implementations, a method for integrated eye surgery may include the steps of: determining a cataract-target region in a lens of the eye; applying cataract-laser pulses to photodisrupt a portion of the determined cataract-target region; determining an astigmatism-target region in a central, mid, or peripheral region of the eye; and applying astigmatism correcting-laser pulses to create one or more incisions in the astigmatism-target region by photodisruption; wherein the steps of the method are performed within an integrated surgical procedure.

In some implementations, the method can include imaging a photodisruption achieved by the cataract-laser pulses; and determining at least portions of an astigmatism-target region in response to the imaged photodisruption.

In some implementations, a multi-purpose ophthalmic surgical system can include a multi-purpose laser, configured to place cataract-laser pulses into a cataract-target target region, and to place astigmatism-laser pulses into an astigmatism-target region; and an imaging system, configured to image a photodisruption caused by at least one of the cataract-laser pulses and the astigmatism-laser pulses.

### BRIEF DESCRIPTION OF THE DRAWINGS

FIG. 1 illustrates an eye.

FIG. 2 illustrates a nucleus of an eye.

FIG. 3 illustrates steps of a photodisruptive method.

FIG. 4 illustrates the application of the surgical laser in step 320a-b.

FIGS. 5A-G illustrate the creation of the corneal and capsular incisions and the insertion of the IOL.

FIGS. 6A-G illustrate various implementations of the cataract surgery integrated with a glaucoma or astigmatism surgery.

FIG. 7 shows an example of an imaging-guided laser surgical system in which an imaging module is provided to provide imaging of a target to the laser control.

FIGS. 8-16 show examples of imaging-guided laser surgical systems with varying degrees of integration of a laser surgical system and an imaging system.

FIG. 17 shows an example of a method for performing laser surgery by suing an imaging-guided laser surgical system.

FIG. 18 shows an example of an image of an eye from an optical coherence tomography (OCT) imaging module.

FIGS. 19A-D show two examples of calibration samples for calibrating an imaging-guided laser surgical system.

FIG. 20 shows an example of attaching a calibration sample material to a patent interface in an imaging-guided laser surgical system for calibrating the system.

FIG. 21 shows an example of reference marks created by a surgical laser beam on a glass surface.

FIG. 22 shows an example of the calibration process and the post-calibration surgical operation for an imaging-guided laser surgical system.

FIGS. 23A-B show two operation modes of an exemplary imaging-guided laser surgical system that captures images of laser-induced photodisruption byproduct and the target issue to guide laser alignment.

FIGS. 24-25 show examples of laser alignment operations in imaging-guided laser surgical systems.

FIG. 26 shows an exemplary laser surgical system based on the laser alignment using the image of the photodisruption byproduct.

### DETAILED DESCRIPTION

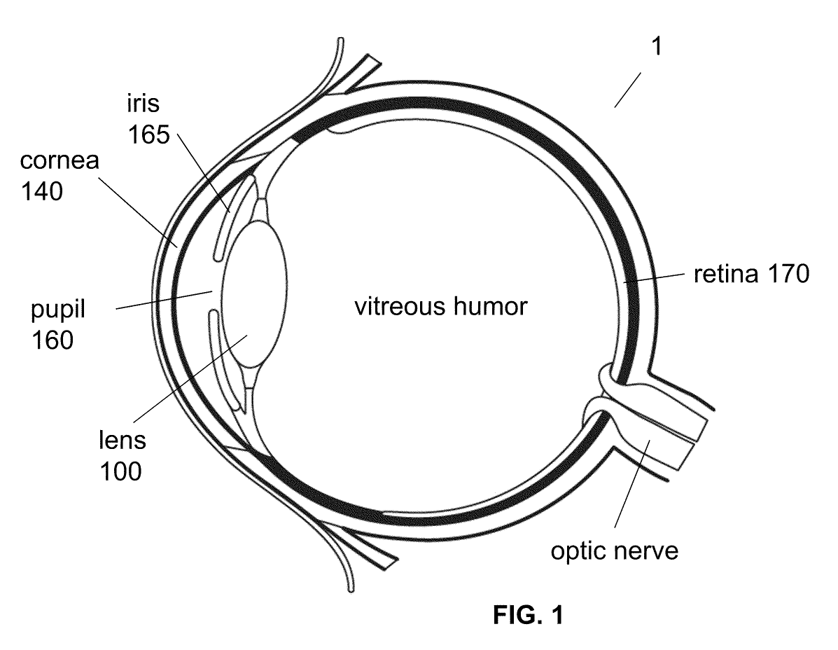

FIG. 1 illustrates the overall structure of the eye 1. The incident light propagates through the optical path which includes the cornea 140, the pupil 160, defined by the iris 165, the lens 100 and the vitreous humor. These optical elements guide the light on the retina 170.

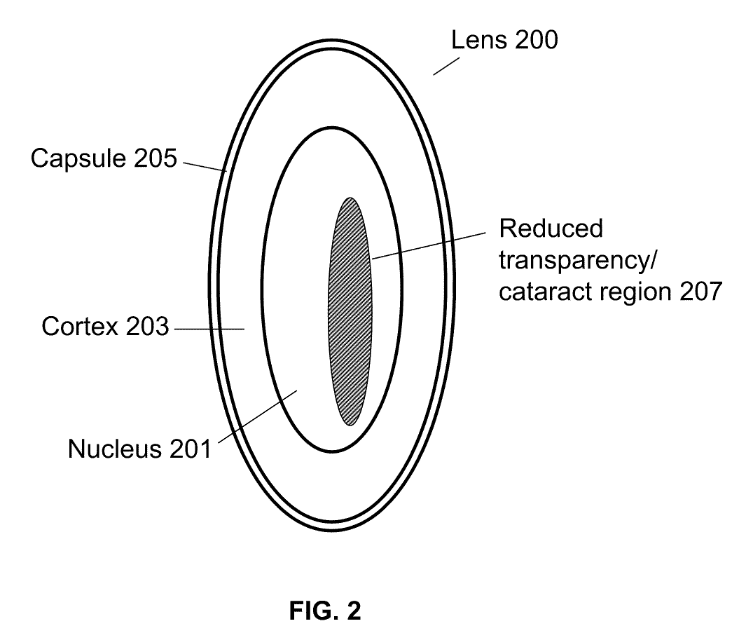

FIG. 2 illustrates a lens 200 in more detail. The lens 200 is sometimes referred to as crystalline lens because of the α, β, and γ crystalline proteins which make up about 90% of the lens. The crystalline lens has multiple optical functions in the eye, including its dynamic focusing capability. The lens is a unique tissue of the human body in that it continues to grow in size during gestation, after birth and throughout life. The lens grows by developing new lens fiber cells starting from the germinal center located on the equatorial periphery of the lens. The lens fibers are long, thin, transparent cells, with diameters typically between 4-7 microns and lengths of up to 12 mm. The oldest lens fibers are located centrally within the lens, forming the nucleus. The nucleus 201 can be further subdivided into embryonic, fetal and adult nuclear zones. The new growth around the nucleus 201, referred to as cortex 203, develops in concentric ellipsoid layers, regions, or zones. Because the nucleus 201 and the cortex 203 are formed at different stages of the human development, their optical properties are distinct. While the lens increases in diameter over time, it may also undergo compaction so that the properties of the nucleus 201 and the surrounding cortex 203 may become even more different (Freel et al BMC Ophthalmology 2003, vol. 3, p. 1).

As a result of this complex growth process, a typical lens 200 includes a harder nucleus 201 with an axial extent of about 2 mm, surrounded by a softer cortex 203 of axial width of 1-2 mm, contained by a much thinner capsule membrane 205, of typical width of about 20 microns. These values may change from person to person to a considerable degree.

Lens fiber cells undergo progressive loss of cytoplasmic elements with the passage of time. Since no blood veins or lymphatics reach the lens to supply its inner zone, with advancing age the optical clarity, flexibility and other functional properties of the lens sometimes deteriorate.

FIG. 2 illustrates, that in some circumstances, including long-term ultraviolet exposure, exposure to radiation in general, denaturation of lens proteins, secondary effects of diseases such as diabetes, hypertension and advanced age, a region of the nucleus 201 can become a reduced transparency region 207. The reduced transparency region 207 is usually a centrally located region of the lens (Sweeney et al Exp Eye res, 1998, vol. 67, p. 587-95). This progressive loss of transparency often correlates with the development of the most common type of cataract in the same region, as well as with an increase of lens stiffness. This process may occur with advancing age in a gradual fashion from the peripheral to the central portion of the lens (Heys et al Molecular Vision 2004, vol. 10, p. 956-63). One result of such changes is the development of presbyopia and cataract that increase in severity and incidence with age.

The removal of this opaque region with reduced transparency, the cataract region, is the objective of the cataract surgery. In many cases this necessitates removal of the entire interior of the lens, leaving only the lens capsule.

As referred to in the background section, a cataract surgery based on phaco-emulsification can suffer various limitations. For example, such an ultrasound-based surgery may produce corneal incisions that are not well controlled in size, shape and location and thus result in lack of self-sealing of the wound. Dealing with uncontrolled incisions may require sutures. The phaco-emulsification technique also requires making a large incision on the capsule, sometimes up to 7 mm. The procedure can leave extensive unintended modifications in its wake: the treated eye can exhibit extensive astigmatism and a residual or secondary refractive or other error. This latter often necessitates a follow-up refractive or other surgery or device. Also, the iris tissue can be torn by the probe, or the procedure can cause a prolapse of iris tissue into the wound. The broken-up lens material may be difficult to access, and the implantation of the IOL challenging. The ultrasound-based surgery may also cause undesired elevated eye pressures due to residual viscoelastic agents that block drainage channels of the eye. In addition, these procedures may lead to non-optimally centered, shaped or sized capsule openings which can cause complications for the removal of lens material and/or limit the precision in positioning and placing IOL in the eye.

The twin causes of the above difficulties and challenges are that the lens break-up is carried out (i) by opening up the eye itself, and (ii) in a large number of separate steps, each requiring the insertion or removal of tools, leaving the eye open between these steps.

These and other limitations and associated risks in cataract surgery using phaco-emulsification have led to development of procedures for treating cataract without making an incision in the eye. For example, U.S. Pat. No. 6,726,679 describes a method to remove lens opacities by directing ultrashort laser pulses to locations of the opacities in the eye. This early method, however, did not appreciate several difficulties with the control of the surgical process. Further, its usefulness was limited for cases when the eye condition was caused by problems other than lens opaqueness. E.g. in the case of a concomitant refractive error, separate procedures were required.

Implementations of the present application describe methods and an apparatus for performing cataract surgery which overcome the above described twin problems. Implementations carry out the lens disruption (i) without opening the eye, and (ii) in a single, integrated procedure. Furthermore, the implementations provide good control of the surgical procedure, reduce the potential for error, minimize the need for additional technical assistance, and enhance the effectiveness of the surgery. The methods and apparatus for cataract surgery described in the present application can be implemented for removing the lens of an eye and integrating the lens removal with other surgical steps, carrying out the entire procedure in a coordinated and efficient manner.

Physical entry into the eye can be avoided by applying photodisruption, utilizing e.g. short pulsed lasers. Operators of eye-surgical lasers are capable of delivering the laser beam to the lens region targeted for fragmentation with high precision. Lens fragmentation based on photodisruption can be implemented in various configurations, such as those described in U.S. Pat. Nos. 4,538,608, 5,246,435, and 5,439,462. The presently described methods and apparatus can be used to allow these and other lens fragmentation methods based on photodisruption to be performed in conjunction with, and integrated with other surgical steps required in cataract surgery including the step to open the eye and/or capsule, the step to remove the fragmented lens material and the step to insert an artificial lens into the void left by the removed fragmented lens.

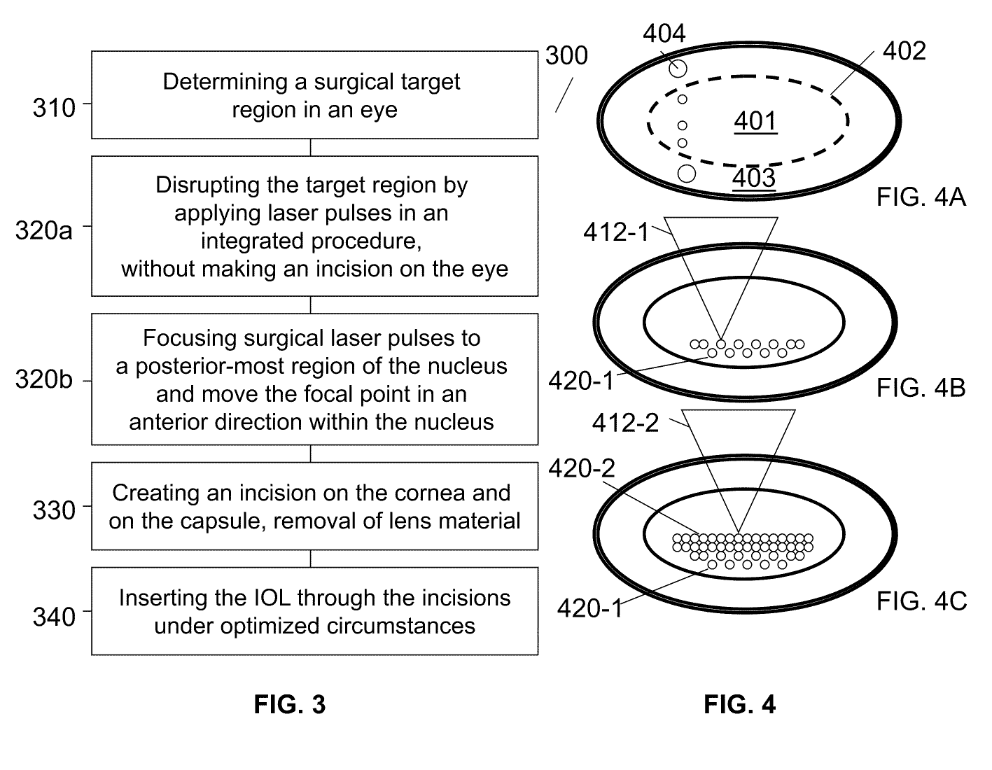

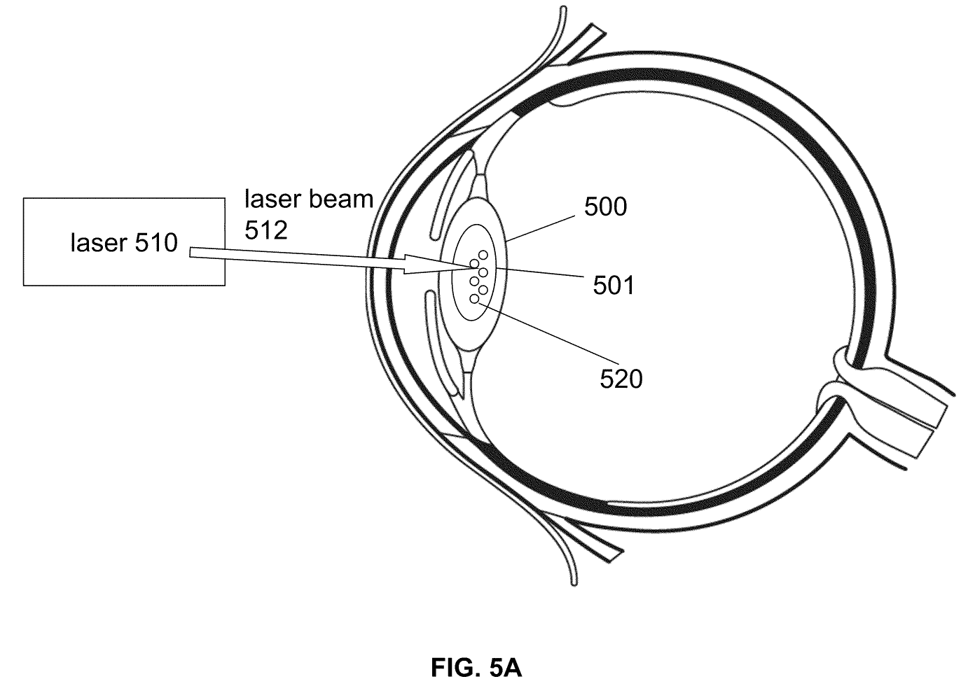

FIGS. 3-4 illustrate that in an implementation 300 of the present methods, the surgical steps for removing a cataract may involve the following.

Step 310 may involve determining a surgical target region in an eye. In several of the described embodiments, the target region can be the nucleus, or a region related to the nucleus which developed a cataract. Other embodiments may target other regions.

FIG. 4A illustrates that in some aspects of step 310 the determining the surgical target region involves determining the boundaries of the target region, such as the boundary 402 of the nucleus. This determination may involve creating a set of probe-bubbles 404 within the lens with laser pulses, and observe their growth, or dynamics. The probe-bubbles grow faster in the cortex region which is softer, whereas the probe-bubbles grow slower in the nucleus, as the nucleus is harder. Other methods can also be practiced to infer the nucleus-boundary 402 from observing the probe bubbles 404, such as ultrasound agitation and measuring a response to it. From the observed growth or dynamics of the probe bubbles 404 the hardness of the surrounding material can be inferred: this is a method well suited to separate the harder nucleus from the softer cortex, thus identifying the boundary of the nucleus.

Step 320a may involve disrupting the target region without having made an incision on the eye. This is achieved by applying laser pulses in an integrated procedure to the target region.

One of the aspects in which step 320a is referred to as an integrated procedure is that step 320a achieves the equivalent effect of five of the steps of the ultrasound-based surgery described above:

(1) Corneal incision and paracentesis; (3) Incision of anterior capsule; (4) Creation of anterior capsulorhexis; (5) Hydrodissection of lens nucleus; (6) Fragmentation of the lens nucleus by mechanical and ultrasound-based methods.

Aspects of step 320a include the following. (i) Since the eye is not opened up for the disruption of the lens, the optical path is not disturbed and the laser beam can be controlled with high precision to hit the intended target region with high precision. (ii) Also, since no physical objects are inserted into incisions of the eye, the incisions do not get torn further by the insertion and extraction of the physical object, in a hard to control manner. (iii) Since the eye is not open during the disruption process, the surgeon does not have to manage the fluids in the open eye, which otherwise would be seeping out and would require replenishment e.g. with injecting viscous fluids, as in step (2) of the ultrasound-based surgery.

In a laser-induced lens fragmentation process, laser pulses ionize a portion of the molecules in the target region. This may lead to an avalanche of secondary ionization processes above a “plasma threshold”. In many surgical procedures a large amount of energy is transferred to the target region in short bursts. These concentrated energy pulses may gasify the ionized region, leading to the formation of cavitation bubbles. These bubbles may form with a diameter of a few microns and expand with supersonic speeds to 50-100 microns. As the expansion of the bubbles decelerates to subsonic speeds, they may induce shockwaves in the surrounding tissue, causing secondary disruption.

Both the bubbles themselves and the induced shockwaves carry out one of the goals of the step 320a: the disruption, fragmentation or emulsification of the nucleus 201 without having made an incision on the capsule 205.

It has been noted that the photodisruption decreases the transparency of the affected region. If the application of the laser pulses starts with focusing the pulses in the frontal or anterior region of the lens and then the focus is moved deeper towards the posterior region, the cavitation bubbles and the accompanying reduced transparency tissue can be in the optical path of the subsequent laser pulses, blocking, attenuating or scattering them. This may diminish the precision and control of the application of the subsequent laser pulses, as well as reduce the energy pulse actually delivered to the deeper posterior regions of the lens. Therefore, the efficiency of laser-based eye surgical procedures can be enhanced by methods in which the bubbles generated by the early laser pulses do not block the optical path of the subsequent laser pulses.

One possible way to preempt the previously generated bubbles from obscuring the optical path of the subsequently applied laser pulses is to first apply the pulses in a posterior-most region of the lens, and then move the focal point towards the anterior regions of the lens.

The technique of U.S. Pat. No. 5,246,435 did not appreciate various difficulties associated with related processes. These problems include that the bubbles generated in the cortex often spread uncontrollably because of the low hardness and the more viscous nature of the cortex. Thus, if a laser is applied to the back of the lens, where the posterior portion of the cortex is, the surgeon will create bubbles which spread rapidly and uncontrollably over large areas, quite possibly obscuring the optical path.

Step 320b is an illustration of an improved way of carrying out step 320a: by focusing surgical laser pulses to a posterior-most region of the nucleus 401 and move the focal point in an anterior direction within the nucleus 401.

FIG. 4B illustrates that embodiments of the present method utilize the approximate knowledge of the boundaries 402 of the nucleus 401, which were determined in step 310. Step 320b preempts the previously generated bubbles from obscuring the optical path of the subsequently applied laser pulses (e.g. by uncontrollably expanding into the cortex 403) by first applying the pulses 412-1 in a posterior-most region 420-1 of the nucleus 401. This is followed by applying the subsequent laser pulses 412-2 to a region 420-2 in the nucleus 401, which is anterior to the region 420-1 where the laser pulses 412-1 were previously applied.

Put another way: the focal point of the laser pulses 412 is moved from a posterior region to an anterior region of the nucleus 401.

An aspect of the steps 320a and 320b is that the laser pulses are applied with a power which is sufficiently strong to achieve the desired photo-disruption of the lens, but not strong enough to cause disruption or other damage in other regions, such as in the retina. Further, the bubbles are placed close enough to cause the desired photo-disruption, but not too close so that the created bubbles coalesce, and form a larger bubble which may grow and spread uncontrollably. The power threshold to achieve disruption may be referred to as “disruption-threshold”, and the power threshold to cause the undesired spreading of gas bubbles maybe referred to as “spread-threshold”.

The above upper and lower thresholds pose limitations on the parameters of the laser pulses such as their power and separation. The duration of the laser pulses may also have analogous disruption- and spread-thresholds. In some implementations the duration may vary in the range of 0.01 picoseconds to 50 picoseconds. In some patients particular results were achieved in the pulse duration range of 100 femtoseconds to 2 picoseconds. In some implementations, the laser energy per pulse can vary between the thresholds of 1 μJ and 25 μJ. The laser pulse repetition rate can vary between the thresholds of 10 kHz and 100 MHz.

The energy, target separation, duration and repeat frequency of the laser pulses can also be selected based on a preoperative measurement of lens optical or structural properties. Alternatively, the selection of the laser energy and the target separation can be based on a preoperative measurement of the overall lens dimensions and the use of an age-dependant algorithm, calculations, cadaver measurements, or databases.

It is noteworthy that laser-disruption techniques developed for other areas of the eye, such as the cornea, cannot be practiced on the lens without substantial modification. One reason for this is that the cornea is a highly layered structure, inhibiting the spread and movement of bubbles very efficiently. Thus, the spread of bubbles poses qualitatively lesser challenges in the cornea than in the softer layers of the lens including the nucleus itself

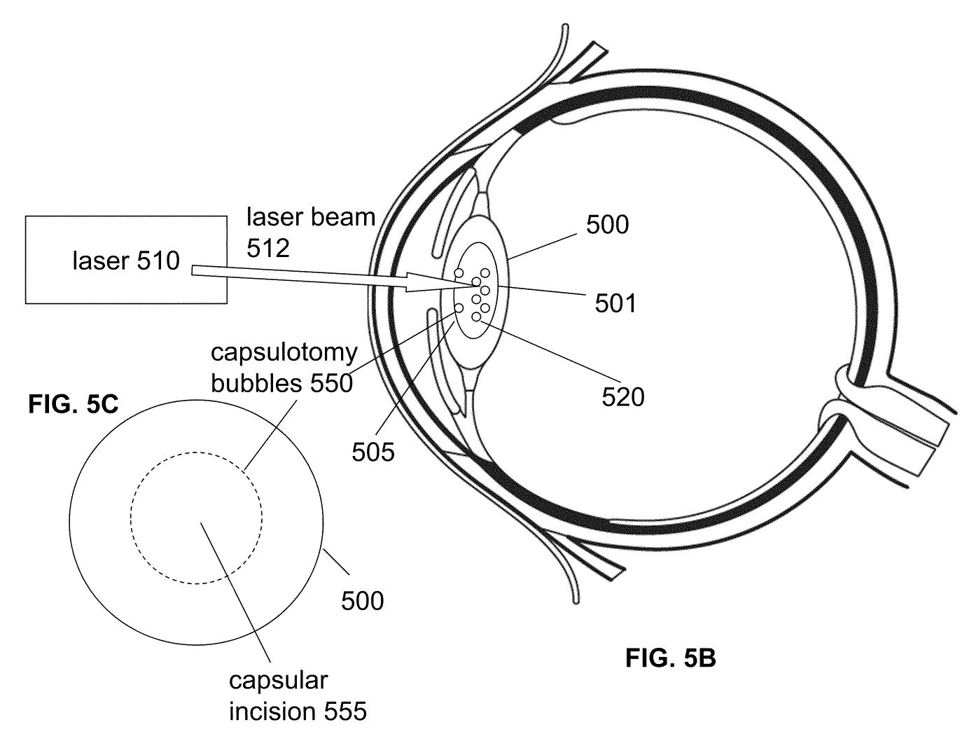

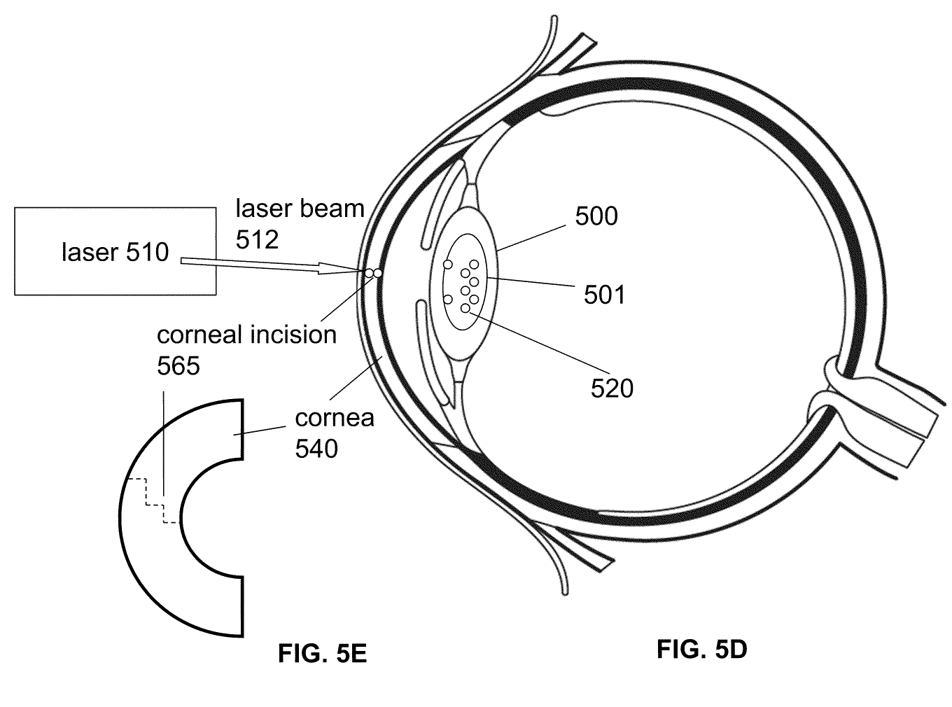

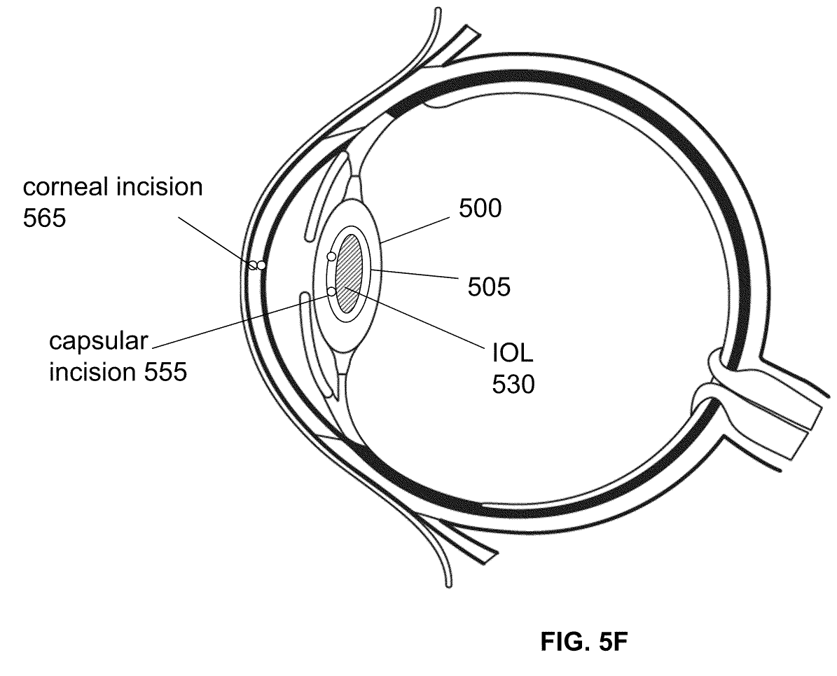

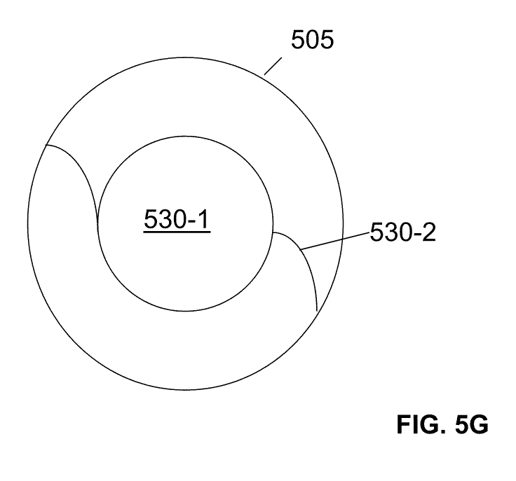

FIG. 5A also illustrates steps 320a-b. In an analogous numbering, laser beam 512 can cause the disruption of the nucleus 501 within the lens 500 by forming bubbles 520, wherein the laser beam 512 is applied with laser parameters between the disruption- and the spread-thresholds, moving its focal point in a posterior-to-anterior direction.

Step 330 may involve making incisions on the cornea and on the capsule. These incisions serve at least two purposes: open a path to for the removal of the disrupted nucleus and the other lens material, and for the subsequent insertion of the IOL.

FIGS. 5B-C illustrate creating an incision on the capsule 505 of the lens 500, sometimes referred to as capsulotomy. In step 330 the laser beam 512 can be focused on the surface of the capsule, such that the created “capsulotomy-bubbles” 550 are sufficient to disrupt the capsule 505, in effect perforating it. FIG. 5B shows a side-view of the eye and FIG. 5C a frontal view of the lens 500 after a ring of the “capsulotomy-bubbles” 550 have been created, defining a capsular incision 555. In some implementations a full circle of these bubbles 550 is formed, and the disc-shaped lid of the capsule, i.e. the capsular incision 555, is simply removed. In other implementations, an incomplete circle is formed on the capsule 505, the lid remains attached to the capsule, and at the end of the procedure the lid maybe restored to its original location.

The disc-like capsular incision 555, defined by the perforation by the capsulotomy-bubbles 550, can then be lifted and removed by a surgical instrument in a later step overcoming minimal resistance from the perforated capsule tissue 505.

FIGS. 5D-E illustrate the creation of an incision on the cornea 540. Laser beam 512 can be applied to create a string of bubbles, which create an incision across the cornea 540. This incision may not be a full circle but a lid, or flap only, which can be re-closed at the end of the procedure.

Again, the application of the surgical laser beam in effect perforates the cornea to define the cornea-lid, so that in a subsequent step the cornea-lid can be easily separated from the rest of the cornea and lifted to allow for physical entry into the eye.

In some implementations, the corneal incision can be a multi-plane, or “valved” incision as shown in the side-view of FIG. 5E (not to scale). Such an incision may be self-sealing and contains the fluid within the eye much better after the surgical procedure is finished. Further, such incisions heal better and stronger, given the more extensive overlap of the corneal tissues, wherein the healing is not hindered by coping with a tear.

These FIGS. 5A-E illustrate well the differences between the incisions in the ultrasound-based surgeries and the presently described photodisruptive surgeries.

The incisions in the ultrasound-based surgeries are made by mechanically tearing the target tissue with a forceps, such as the cornea and the capsule: the so-called curvilinear capsulorhexis technique. Further, the side of the incisions in the ultrasound-based surgeries are repeatedly impacted by the in and out movement of various mechanical devices. For these reasons, the contours of the incisions cannot be controlled too well, and the incisions cannot be made in the above described self-sealing manner. Thus, the ultrasound-based method has poorer size-control and lacks the self-sealing aspect of the multi-plane incisions, which are possible with the photodisruptive treatments.

This has been demonstrated in testing procedures when the creation of a nominally 5 mm opening was attempted by both procedures. The incision created by mechanical tearing had a diameter of 5.88 mm, with a variance of 0.73 mm. In contrast, with the photodisruptive method described here an opening with diameter 5.02 mm was achieved with a variance of 0.04 mm.

These test results demonstrate the qualitatively higher precision of the photodisruptive method. The importance of this difference can be appreciated e.g. from the fact that if an astigmatic correcting incision of a cornea is off only by 10-20%, this will negate or even counteract much of its intended affect, possibly requiring a follow-up surgery.

Further, the moment the cornea is opened up by an incision in the ultrasound-based method, the “aqueous humor of the anterior chamber”, i.e. the fluid content of the eye, starts escaping, in effect, the fluid starts dripping out of the eye.

This loss of fluid has negative consequences, since the aqueous humor plays an essential role in sustaining the structural integrity of the eye, by propping it up, somewhat akin to the water in a water-filled balloon.

Therefore, considerable effort has to be spent to continuously replenish the fluid escaping from the eye. In ultrasound-based surgeries a complex, computer-controlled system monitors and oversees this fluid-management. However, this task requires considerable skill from the surgeon herself.

In contrast, implementations of the present method do not open up the eye to achieve photodisruption. For this reason, fluid-management is not a task during the photodisruption of the lens, thus requiring less skill from the surgeon and less complex equipment.

Referring again to FIG. 3, step 330 also includes the removal of the fragmented, disrupted, emulsified or otherwise modified nucleus and other lens material, such as the more fluid cortex. This removal is typically carried out by inserting an aspiration probe through the corneal and capsular incisions, and aspirating the material.

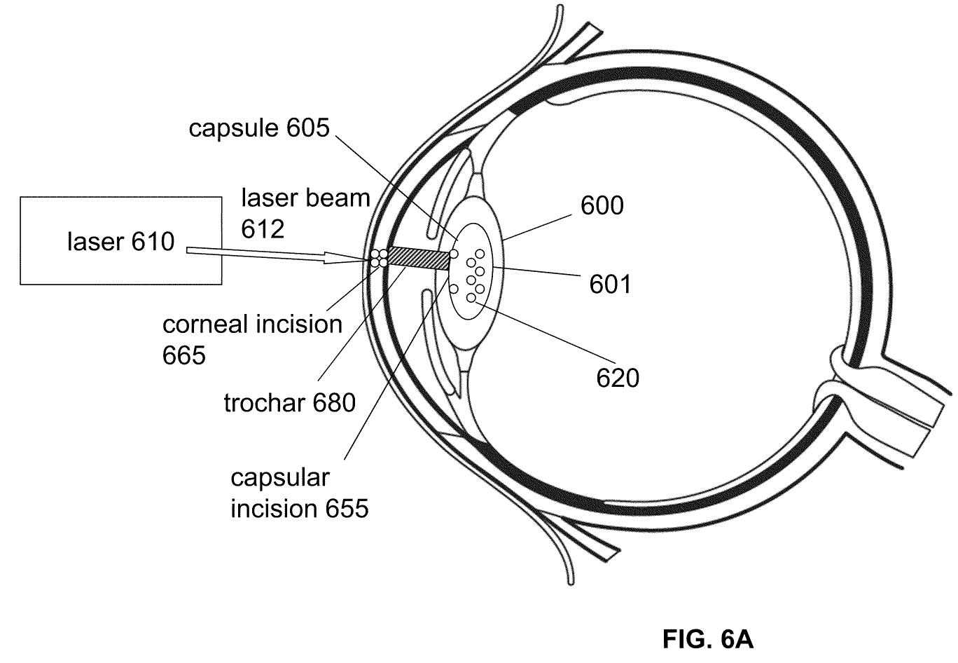

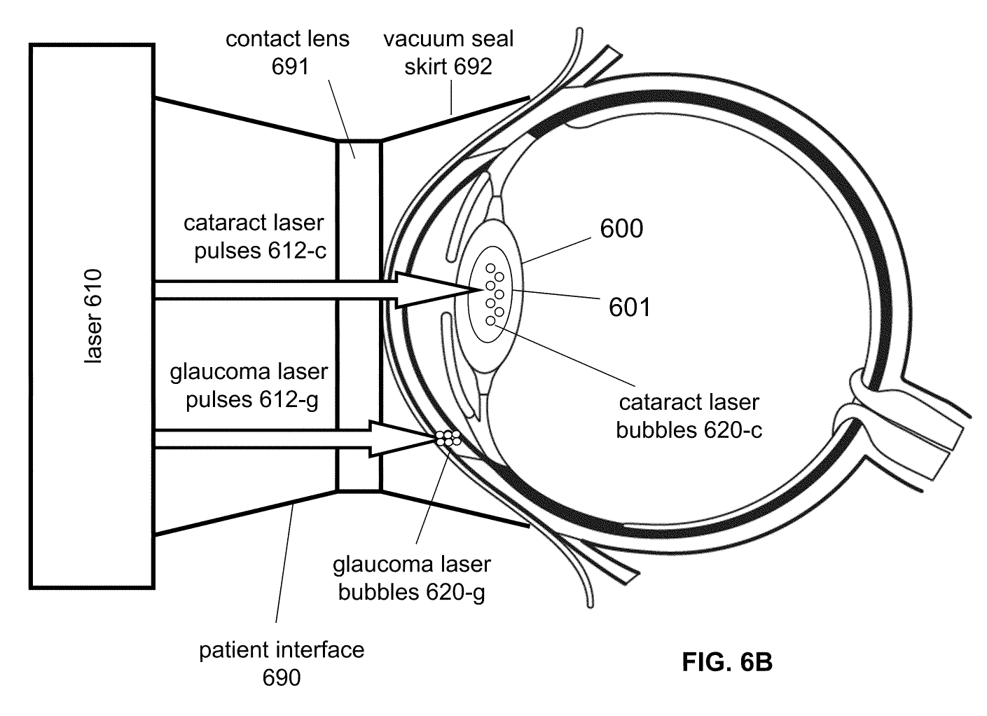

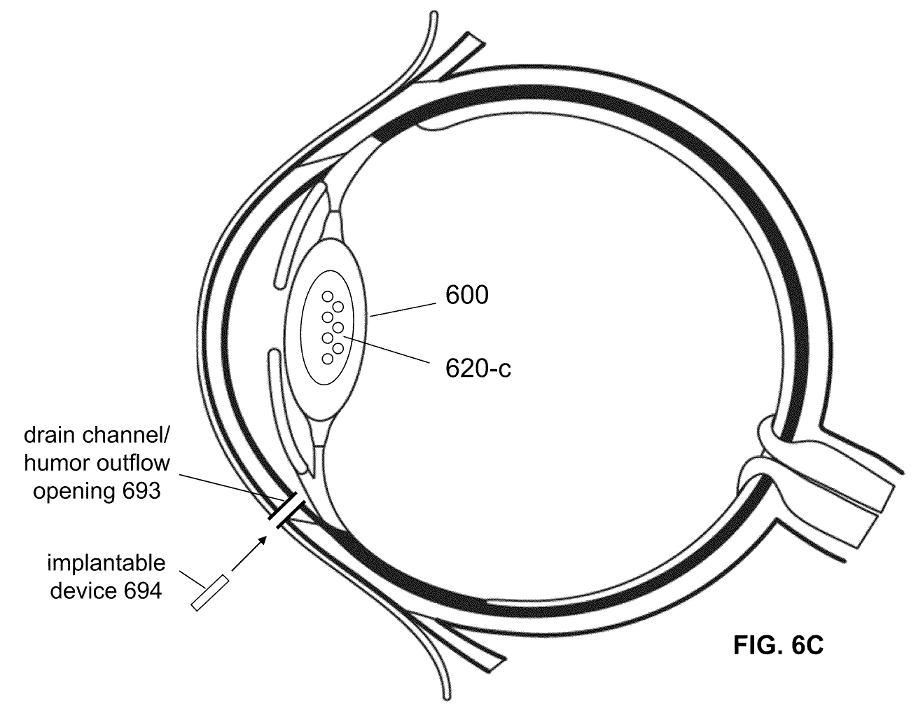

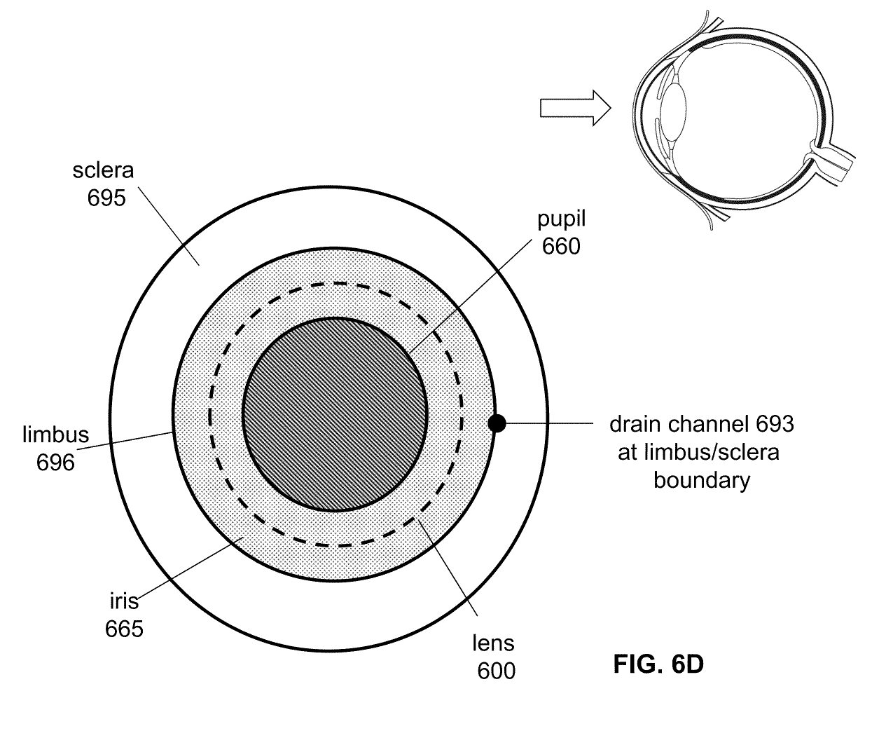

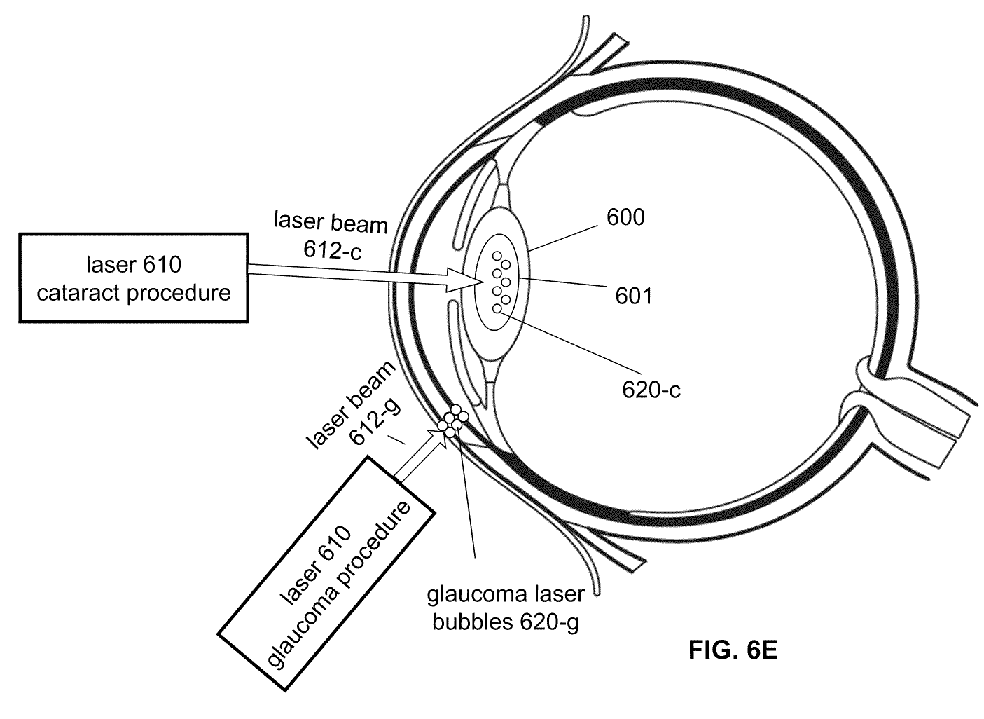

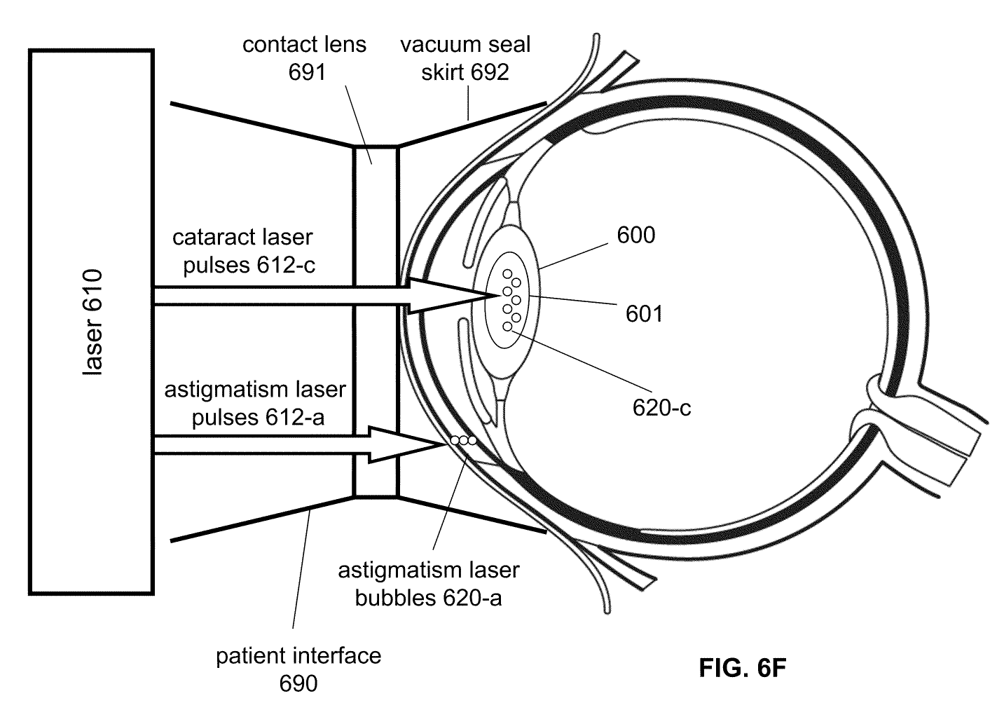

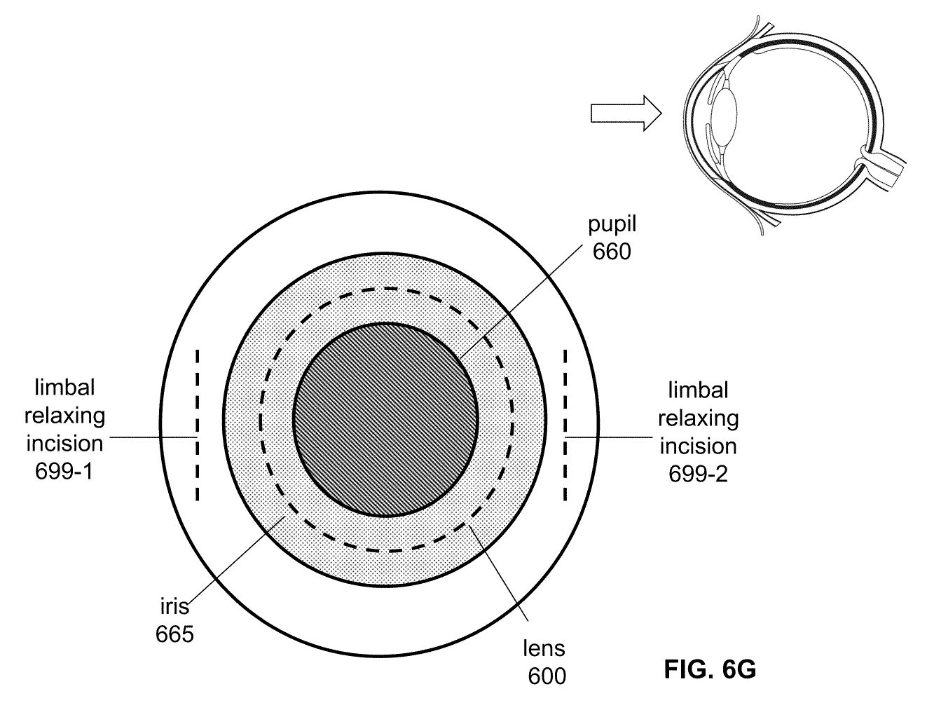

FIG. 5F illustrates that step 340 may include inserting an intra ocular lens (IOL) 530 into the lens capsule 505, to replace the disrupted original lens. The previously created corneal and capsular incisions may serve as entry ports for the IOL insertion. In the present method 300 the incisions were not made to accommodate the phaco-probe. Therefore, the positioning of the incisions, their centeredness and angle can be optimized for the insertion of the IOL 530. The capsulotomy-bubbles 550 and the corneal incision 555 can be all deployed to optimize the insertion of the IOL 530. Then the IOL 530 can be inserted and the opening in the cornea re-closed or left to self-seal. The lens capsule 505 typically wraps around and accommodates the IOL 530 without much intervention. In cases when the capsular incision is large, often a centered location is chosen for the incision. In cases when the capsular incision is small, as in the case of FIG. 6 below, an off-center incision may be used.

FIG. 5G illustrates that the intra ocular lens 530 can contain an “optic” portion 530-1, which can be essentially a lens and a “haptic” portion 530-2, which can be a wide variety of devices or arrangements, whose functions include holding the optic portion 530-1 in a desired position inside the capsule 505. In some implementations, the optic portion 530-1 can be considerably smaller than a diameter of the capsule 505, necessitating such holding “haptic” portions. FIG. 5G shows an embodiment where the haptic portion 530-2 includes two spiraling arms.

In some embodiments of the present system an optic-haptic junction is engaged by making one or more incisions in an anterior capsule.

In some implementations, the lens capsule 505 is inflated during the insertion of the IOL so that the haptic portion 530-2 can be placed optimally. For example, the haptic portion 530-2 can be placed into the most peripheral recesses of the capsule 505, to optimize centration and anterior-posterior localization of the optic portion 530-1.

In some implementations, the lens capsule 505 is deflated following the insertion of the IOL to bring the anterior and posterior portion of the capsule 505 together in a controlled manner to optimize centration and anterior-posterior localization of the optic portion 530-1.

In some implementations of the above described eye surgery peripheral areas of the lens are accessed optically via an angled mirror.

In some cases it may occur that peripheral regions of the lens 600 may not be accessible optically. In some implementations of the present methods these areas can be fragmented or dissolved by means other than photodisruption, including ultrasound, heated water or aspiration.

FIG. 6A illustrates an implementation which shares many elements with FIGS. 3-5F, analogously numbered, which will not be repeated here. In addition, the implementation of FIG. 6A contains a trochar 680. The trochar 680, which is essentially a suitably shaped cylinder, can be inserted through the corneal incision 665, all the way into the lens capsule 605 through the capsular incision 655. In some cases the diameter of the trochar can be about 1 mm, in others in the range of 0.1-2 mm.

This trochar 680 can offer improved control in various stages of the above photodisruptive process. The trochar 680 can be used for the fluid management, as it creates a controlled channel to move fluids in an out. In some embodiments it is possible to deploy the trochar 680 in an essentially watertight manner into the corneal incision 665 and the capsular incision 655. In these embodiments, there is minimal seepage outside the trochar 680 and thus the need for managing the fluids outside the trochar 680 is minimal too.

Further, instruments can be moved in an out in a more controlled, safer manner through the trochar 680. Also, the photodisrupted nucleus and other lens material can be more safely removed, in a well controlled manner. Finally, the IOL can be inserted through the trochar 680, as some IOLs can be folded up to have a maximal size of 2 mm or less. These IOLs can be moved through the trochar 680 having a diameter slightly larger than that of the folded IOL. Once in place, the IOLs can be unfolded or unpacked inside the capsule 605 of the lens 600. The IOLs can be also properly aligned so that they will be located centrally and without an undesired tilt inside the capsule 605 of the lens 600. Further, trochar-based surgical procedures require the creation of quite small incisions, of the order of 2 mm, instead of the 7 mm type incisions, used in phaco-emulsification.

In general, the trochar 680 maintains a partially or fully insulated and controlled space of operations. Once the operations are concluded, the trochar 680 can be removed and the corneal self-sealing incision 665 can heal effectively and securely. By using this method the photodisruptive process can restore the vision of the patient to a maximum possible degree.

In sum, embodiments of the described photodisruptive method are capable and configured to carry out the steps of photodisruption of the nucleus of the lens of an eye, or any other target area (i) without creating an opening in the eye; and (ii) with a single integrated process, instead of requiring numerous steps carried out by different devices, and high skill from the surgeon.

One implementation of the present apparatus for cataract surgery can maintain the ocular volume by eliminating or reducing the need for viscoelastics and can provide easier placement of an IOL in an inflated, minimally disturbed capsular bag to optimize placement and maintenance of IOL in optimally centered and non-tilted position. This process can increase the optical and/or refractive predictability and functioning of the eye after the intervention. This process also reduces the need for surgical assistance and provides an opportunity for operative efficiencies, such as dividing the procedure into two parts that can be performed under different levels of sterility, in different rooms or even at different times.

For example, the laser procedure can be performed in a lower overhead, nonsterile environment at a first time, with the lens removal and IOL placement performed in a traditional sterile environment, such as an operating room at a later time. Alternatively, since the level of skill and support required for the lens removal and IOL placement is reduced due to the use of photodisruption, the level of requirements for the venue may also be reduced, with resulting savings in cost, time or increased convenience (such as the ability to perform procedures in a procedure room setting similar to LASIK surgery).

The above discussed cataract eye disease often coexists with another ailment of the eye, glaucoma. Glaucoma is associated with diseases of the optic nerve, resulting from an excess intraocular pressure (TOP) of the aqueous humor. Draining a suitable amount of aqueous humor may result in a reduction of the excess IOP and a reversal of the diseases of the optic nerve. Creating incisions in a peripheral ophthalmic region by the application of surgical lasers may release the IOP on a one-time basis or may create a permanent drain channel to stabilize the IOP at a lower level. Thus, ophthalmic laser surgery constitutes a promising approach to treat glaucoma.

In patients having cataract and glaucoma, it may be beneficial to treat both conditions at the same time. And even in cases when the procedures are not performed concurrently, there may be a benefit in coordinating the incisions for each procedure to minimize the potential for complications and maximize the successful outcome of each procedure.

FIGS. 6B-D illustrate implementations of integrated ophthalmic surgical procedures that perform cataract and glaucoma procedures either concurrently or in an integrated or coordinated manner.

FIG. 6B illustrates that in an integrated ophthalmic procedure a surgical laser 610 can be utilized to apply a set of cataract procedure laser pulses 612-c into the nucleus 601 of the lens 600 to form a set of cataract procedure laser bubbles 620-c. Before, after or concurrently with the cataract procedure, the surgical laser 610 may apply a set of glaucoma procedure laser pulses 612-g to a peripheral region of the eye, such as the sclera, the limbal region, an ocular angle portion, or the iris root. These glaucoma procedure laser pulses 612-g may be part of any known glaucoma procedure, including trabeculoplasty, iridotomy or iridectomy, among others. In any one of these procedures a set of glaucoma procedure laser bubbles 620-g are generated in a peripheral ophthalmic region to create one or more incisions or openings according to various patterns.

FIG. 6C illustrates that in some implementations these incisions or openings can eventually form a drain channel or humor outflow opening 693. In some embodiments, an implantable device 694 can be inserted into the drain channel to regulate the outflow. The implantable device 694 can be a simple drain tube, or can contain a pressure controller or valve. Its shape can be straight or may have turns, corners or elbows.

In any one of these implementations, the drain channel 693 or the implantable device 694 can connect an anterior chamber of the eye to a surface of the eye, thus facilitating the reduction of the intra ocular pressure.

FIG. 6B illustrates an implementation of the integrated ophthalmic procedure where the surgical laser 610 has a patient interface 690, including a contact lens 691 which can be a flat applanation plate or a curved lens, as well as a vacuum seal skirt 692 that applies a partial vacuum to at least partially immobilize the eye for the procedure. If the patient interface 690 is suitably sized then the surgical laser does not need to be repositioned or adjusted. In these embodiments, an x-y or x-y-z scanning system may be able to deflect or direct the surgical laser sufficiently to reach the peripheral ophthalmic regions of the glaucoma procedure.

In integrated procedures, the contact lens 691 can be changed from a contact lens 691-c, optimized for cataract procedures to another contact lens 691-g, optimized for glaucoma procedures.

The sclera scatters the incident laser light strongly, evidenced, for example, by its bright white color. Therefore, lasers at most wavelengths are not particularly efficient to cut through the sclera and form the drain channel 693. Restated differently, to create a trans-scleral incision, the laser beams may have to have such high energies that can cause excessive disruption in the ophthalmic tissue.

To address this challenge, in some integrated systems specific wavelengths λ-g are identified at which the absorption and scattering by the sclera has a dip, minimum, or gap. Lasers with such wavelengths can be useful to form the drain channel 693 in the sclera. However, these glaucoma-specific wavelengths λ-g may not be particularly suitable for the cataract procedures, which may work best at different λ-c wavelengths.

Therefore, in some implementations an operating wavelength of the surgical laser 610 may be changeable from a cataract-optimized λ-c value to a glaucoma-optimized λ-g value. In other implementations, separate lasers can be utilized: one for the cataract procedure operating at the wavelength λ-c, and one for the glaucoma procedure operating at the wavelength λ-g.

However, changing the operating wavelength of the surgical laser might be challenging, and having a system with two different lasers may pose difficulties for optimizing the optical performance and keeping the system costs competitive.

FIG. 6D illustrates that some implementations address these issues by including a single wavelength laser and direct it to regions that are optimized for the competing and partially contradictory requirements of keeping the scattering by the target region low while minimizing the perturbation of the optical pathway.

One such optimized region can be, for example, a boundary region between the sclera 695 and the limbus 696. This limbus/sclera boundary region may scatter the laser beam less than the sclera itself, thus allowing the use of a single laser for both the glaucoma and the cataract procedures with a wavelength selected to perform cataract procedures sufficiently well but not necessarily to minimize scattering and absorption by the sclera. At the same time, the drain channel 693 in this limbus/sclera boundary region can be in a sufficiently peripheral region so that it perturbs the optical pathway and thus the vision of the patient only to a minimal degree. Typically, target selection farthest from the optical axis of the eye can be useful in this aspect. Other target regions may also represent good compromises between the requirements of the glaucoma and the cataract surgeries, such as the intersection of the cornea and the limbus.

Besides its location, the direction of the drain channel 693 may also impact the efficiency of the formation of the drain channel 693. For example, the drain channel 693 can be directed in a way that is not necessarily perpendicular to the surface of the eye, but rather, is chosen to go through those regions of the sclera that scatter least and thus require only limited energy laser pulses.

FIG. 6E illustrates implementations of the integrated ophthalmic procedure where the surgical laser 610 is either adjusted between the cataract procedure and the glaucoma procedure, or where in fact separate lasers are utilized for the two procedures.

The precision of these procedures can be enhanced by imaging the surgical regions. For an integrated cataract-glaucoma procedure an imaging system may be integrated with the laser surgical system as described below. The imaging system can be configured to image the lens 600, cornea 140, limbal, scleral or ocular angle portions of the eye. The images can be analyzed to coordinate the formation of incisions for the cataract procedure and the glaucoma procedure so that the performance of the integrated procedures is optimized.

In implementations when the two procedures are performed sequentially, an imaging step can be performed after the first procedure to image the bubbles formed and photodisruption achieved in the course of the first procedure. This image can aid and guide the placement of the laser pulses of the second procedure.

In particular, if the cataract procedure is performed first, a subsequent imaging step can be performed to image the photodisruption caused by the cataract procedure laser pulses 612-c. This image can be used to select the target regions where the glaucoma procedure laser pulses 612-g will be directed to. And in reverse, if the glaucoma procedure is performed first, a subsequent imaging step can be performed to image the photodisruption caused by the glaucoma procedure laser pulses 612-g. This image can be used to select the target regions where the cataract procedure laser pulses 612-c will be directed to.

In an analogous embodiment, in patients having cataract and astigmatism, it may also be beneficial to treat both conditions at the same time. And even in cases when the procedures are not performed concurrently, there may be a benefit in coordinating the incisions for each procedure to minimize the potential for complications and maximize the successful outcome of each procedure.

FIGS. 6F-G illustrate implementations of integrated ophthalmic surgical procedures that perform cataract and astigmatism procedures either concurrently or in an integrated or coordinated manner.

FIG. 6F illustrates that in an integrated ophthalmic procedure a surgical laser 610 can be utilized to apply a set of cataract procedure laser pulses 612-c into the nucleus 601 of the lens 600 to form a set of cataract procedure laser bubbles 620-c. Before, after or concurrently with the cataract procedure, the surgical laser 610 may apply a set of astigmatism procedure laser pulses 612-a to a central, mid or peripheral cornea, or the limbal region. These astigmatism procedure laser pulses 612-a may be part of any known astigmatism procedure, including astigmatic keratotomy, limbal relaxing incision or corneal wedge resection, among others. In any one of these procedures a set of astigmatism procedure laser bubbles 620-a may be generated to create one or more incisions or openings according to various patterns to reduce a type of corneal astigmatism.

FIG. 6G illustrates an implementation of the integrated ophthalmic procedure with a frontal view of the eye. As part of the astigmatism procedure, a limbal relaxing incision 699-1 and 699-2 may be created in a peripheral, limbal region. When designed with the use of diagnostic optical measurements, such limbal relaxing regions can be helpful to relax an astigmatism of the eye.

In other aspects, the just-described integrated astigmatism-cataract procedure can have several features analogous to the earlier integrated glaucoma-cataract procedure.

These features include (a) using a patient interface with a contact lens to at least partially immobilize the eye for the procedure; (b) using an x-y or x-y-z scanning systems to direct the laser beam according to an astigmatic pattern; (c) changing the contact lens between the procedures; (d) changing the wavelength of the laser between procedures, or using different lasers for the procedures; (e) selecting the location of the astigmatism procedure by optimizing the requirements of minimal scattering by the sclera while placing the astigmatism-related incisions to perturb the optical pathway to the smallest degree; and (f) adjusting a position or a direction of the laser between procedures.

Further, the precision of the integrated cataract-astigmatism procedure can be also enhanced by imaging the surgical regions by integrating an imaging system with the laser surgical system. The imaging system can be configured to image the lens 600, cornea 140, limbal, scleral or ocular angle portions of the eye. The images can be analyzed to coordinate the formation of incisions for the cataract procedure and the astigmatism procedure so that the performance of the integrated procedures is optimized.

In implementations when the two procedures are performed sequentially, an imaging step can be performed after the first procedure to image the bubbles formed and photodisruption achieved in the course of the first procedure. This image can aid and guide the placement of the laser pulses of the second procedure.

In particular, if the cataract procedure is performed first, a subsequent imaging step can be performed to image the photodisruption caused by the cataract procedure laser pulses 612-c. This image can be used to select the target regions where the astigmatism procedure laser pulses 612-a will be directed to. And in reverse, if the astigmatism procedure is performed first, a subsequent imaging step can be performed to image the photodisruption caused by the astigmatism procedure laser pulses 612-a. This image can be used to select the target regions where the cataract procedure laser pulses 612-c will be directed to.

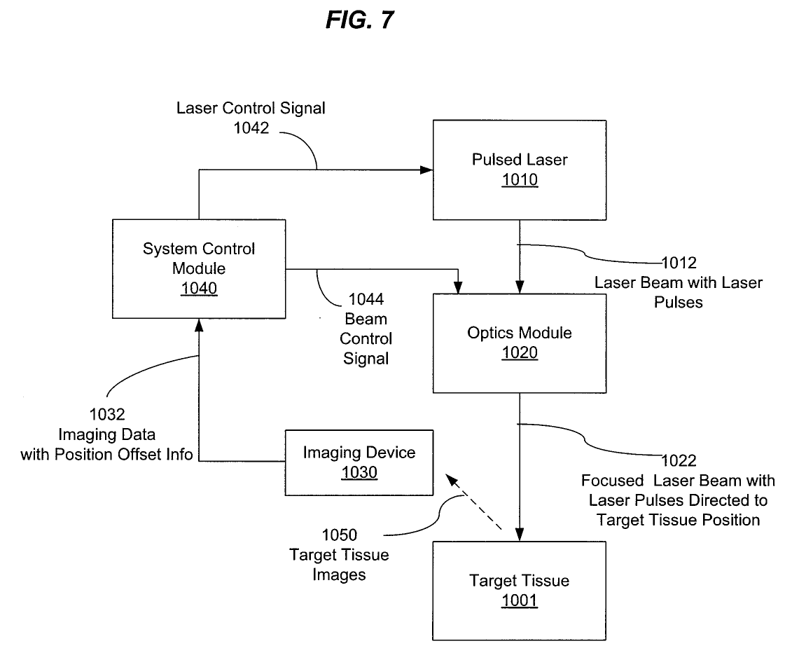

FIGS. 7-26 illustrate embodiments of a laser surgery system in relation to the above photodisruptive laser treatment.

One important aspect of laser surgical procedures is precise control and aiming of a laser beam, e.g., the beam position and beam focusing. Laser surgery systems can be designed to include laser control and aiming tools to precisely target laser pulses to a particular target inside the tissue. In various nanosecond photodisruptive laser surgical systems, such as the Nd:YAG laser systems, the required level of targeting precision is relatively low. This is in part because the laser energy used is relatively high and thus the affected tissue area is also relatively large, often covering an impacted area with a dimension in the hundreds of microns. The time between laser pulses in such systems tend to be long and manual controlled targeting is feasible and is commonly used. One example of such manual targeting mechanisms is a biomicroscope to visualize the target tissue in combination with a secondary laser source used as an aiming beam. The surgeon manually moves the focus of a laser focusing lens, usually with a joystick control, which is parfocal (with or without an offset) with their image through the microscope, so that the surgical beam or aiming beam is in best focus on the intended target.

Such techniques designed for use with low repetition rate laser surgical systems may be difficult to use with high repetition rate lasers operating at thousands of shots per second and relatively low energy per pulse. In surgical operations with high repetition rate lasers, much higher precision may be required due to the small effects of each single laser pulse and much higher positioning speed may be required due to the need to deliver thousands of pulses to new treatment areas very quickly.

Examples of high repetition rate pulsed lasers for laser surgical systems include pulsed lasers at a pulse repetition rate of thousands of shots per second or higher with relatively low energy per pulse. Such lasers use relatively low energy per pulse to localize the tissue effect caused by laser-induced photodisruption, e.g., the impacted tissue area by photodisruption on the order of microns or tens of microns. This localized tissue effect can improve the precision of the laser surgery and can be desirable in certain surgical procedures such as laser eye surgery. In one example of such surgery, placement of many hundred, thousands or millions of contiguous, nearly contiguous or pulses separated by known distances, can be used to achieve certain desired surgical effects, such as tissue incisions, separations or fragmentation.

Various surgical procedures using high repetition rate photodisruptive laser surgical systems with shorter laser pulse durations may require high precision in positioning each pulse in the target tissue under surgery both in an absolute position with respect to a target location on the target tissue and a relative position with respect to preceding pulses. For example, in some cases, laser pulses may be required to be delivered next to each other with an accuracy of a few microns within the time between pulses, which can be on the order of microseconds. Because the time between two sequential pulses is short and the precision requirement for the pulse alignment is high, manual targeting as used in low repetition rate pulsed laser systems may be no longer adequate or feasible.

One technique to facilitate and control precise, high speed positioning requirement for delivery of laser pulses into the tissue is attaching a applanation plate made of a transparent material such as a glass with a predefined contact surface to the tissue so that the contact surface of the applanation plate forms a well-defined optical interface with the tissue. This well-defined interface can facilitate transmission and focusing of laser light into the tissue to control or reduce optical aberrations or variations (such as due to specific eye optical properties or changes that occur with surface drying) that are most critical at the air-tissue interface, which in the eye is at the anterior surface of the cornea. Contact lenses can be designed for various applications and targets inside the eye and other tissues, including ones that are disposable or reusable. The contact glass or applanation plate on the surface of the target tissue can be used as a reference plate relative to which laser pulses are focused through the adjustment of focusing elements within the laser delivery system. This use of a contact glass or applanation plate provides better control of the optical qualities of the tissue surface and thus allow laser pulses to be accurately placed at a high speed at a desired location (interaction point) in the target tissue relative to the applanation reference plate with little optical distortion of the laser pulses.

One way for implementing an applanation plate on an eye is to use the applanation plate to provide a positional reference for delivering the laser pulses into a target tissue in the eye. This use of the applanation plate as a positional reference can be based on the known desired location of laser pulse focus in the target with sufficient accuracy prior to firing the laser pulses and that the relative positions of the reference plate and the individual internal tissue target must remain constant during laser firing. In addition, this method can require the focusing of the laser pulse to the desired location to be predictable and repeatable between eyes or in different regions within the same eye. In practical systems, it can be difficult to use the applanation plate as a positional reference to precisely localize laser pulses intraocularly because the above conditions may not be met in practical systems.

For example, if the crystalline lens is the surgical target, the precise distance from the reference plate on the surface of the eye to the target tends to vary due to the presence of collapsible structures, such as the cornea itself, the anterior chamber, and the iris. Not only is their considerable variability in the distance between the applanated cornea and the lens between individual eyes, but there can also be variation within the same eye depending on the specific surgical and applanation technique used by the surgeon. In addition, there can be movement of the targeted lens tissue relative to the applanated surface during the firing of the thousands of laser pulses required for achieving the surgical effect, further complicating the accurate delivery of pulses. In addition, structure within the eye may move due to the build-up of photodisruptive byproducts, such as cavitation bubbles. For example, laser pulses delivered to the crystalline lens can cause the lens capsule to bulge forward, requiring adjustment to target this tissue for subsequent placement of laser pulses. Furthermore, it can be difficult to use computer models and simulations to predict, with sufficient accuracy, the actual location of target tissues after the applanation plate is removed and to adjust placement of laser pulses to achieve the desired localization without applanation in part because of the highly variable nature of applanation effects, which can depend on factors particular to the individual cornea or eye, and the specific surgical and applanation technique used by a surgeon.

In addition to the physical effects of applanation that disproportionably affect the localization of internal tissue structures, in some surgical processes, it may be desirable for a targeting system to anticipate or account for nonlinear characteristics of photodisruption which can occur when using short pulse duration lasers. Photodisruption is a nonlinear optical process in the tissue material and can cause complications in beam alignment and beam targeting. For example, one of the nonlinear optical effects in the tissue material when interacting with laser pulses during the photodisruption is that the refractive index of the tissue material experienced by the laser pulses is no longer a constant but varies with the intensity of the light. Because the intensity of the light in the laser pulses varies spatially within the pulsed laser beam, along and across the propagation direction of the pulsed laser beam, the refractive index of the tissue material also varies spatially. One consequence of this nonlinear refractive index is self-focusing or self-defocusing in the tissue material that changes the actual focus of and shifts the position of the focus of the pulsed laser beam inside the tissue. Therefore, a precise alignment of the pulsed laser beam to each target tissue position in the target tissue may also need to account for the nonlinear optical effects of the tissue material on the laser beam. In addition, it may be necessary to adjust the energy in each pulse to deliver the same physical effect in different regions of the target due to different physical characteristics, such as hardness, or due to optical considerations such as absorption or scattering of laser pulse light traveling to a particular region. In such cases, the differences in non-linear focusing effects between pulses of different energy values can also affect the laser alignment and laser targeting of the surgical pulses.

Thus, in surgical procedures in which non superficial structures are targeted, the use of a superficial applanation plate based on a positional reference provided by the applanation plate may be insufficient to achieve precise laser pulse localization in internal tissue targets. The use of the applanation plate as the reference for guiding laser delivery may require measurements of the thickness and plate position of the applanation plate with high accuracy because the deviation from nominal is directly translated into a depth precision error. High precision applanation lenses can be costly, especially for single use disposable applanation plates.

The techniques, apparatus and systems described in this document can be implemented in ways that provide a targeting mechanism to deliver short laser pulses through an applanation plate to a desired localization inside the eye with precision and at a high speed without requiring the known desired location of laser pulse focus in the target with sufficient accuracy prior to firing the laser pulses and without requiring that the relative positions of the reference plate and the individual internal tissue target remain constant during laser firing. As such, the present techniques, apparatus and systems can be used for various surgical procedures where physical conditions of the target tissue under surgery tend to vary and are difficult to control and the dimension of the applanation lens tends to vary from one lens to another. The present techniques, apparatus and systems may also be used for other surgical targets where distortion or movement of the surgical target relative to the surface of the structure is present or non-linear optical effects make precise targeting problematic. Examples for such surgical targets different from the eye include the heart, deeper tissue in the skin and others.

The present techniques, apparatus and systems can be implemented in ways that maintain the benefits provided by an applanation plate, including, for example, control of the surface shape and hydration, as well as reductions in optical distortion, while providing for the precise localization of photodisruption to internal structures of the applanated surface. This can be accomplished through the use of an integrated imaging device to localize the target tissue relative to the focusing optics of the delivery system. The exact type of imaging device and method can vary and may depend on the specific nature of the target and the required level of precision.

An applanation lens may be implemented with another mechanism to fix the eye to prevent translational and rotational movement of the eye. Examples of such fixation devices include the use of a suction ring. Such fixation mechanism can also lead to unwanted distortion or movement of the surgical target. The present techniques, apparatus and systems can be implemented to provide, for high repetition rate laser surgical systems that utilize an applanation plate and/or fixation means for non-superficial surgical targets, a targeting mechanism to provide intraoperative imaging to monitor such distortion and movement of the surgical target.

Specific examples of laser surgical techniques, apparatus and systems are described below to use an optical imaging module to capture images of a target tissue to obtain positioning information of the target tissue, e.g., before and during a surgical procedure. Such obtained positioning information can be used to control the positioning and focusing of the surgical laser beam in the target tissue to provide accurate control of the placement of the surgical laser pulses in high repetition rate laser systems. In one implementation, during a surgical procedure, the images obtained by the optical imaging module can be used to dynamically control the position and focus of the surgical laser beam. In addition, lower energy and shot laser pulses tend to be sensitive to optical distortions, such a laser surgical system can implement an applanation plate with a flat or curved interface attaching to the target tissue to provide a controlled and stable optical interface between the target tissue and the surgical laser system and to mitigate and control optical aberrations at the tissue surface.

As an example, FIG. 7 shows a laser surgical system based on optical imaging and applanation. This system includes a pulsed laser 1010 to produce a surgical laser beam 1012 of laser pulses, and an optics module 1020 to receive the surgical laser beam 1012 and to focus and direct the focused surgical laser beam 1022 onto a target tissue 1001, such as an eye, to cause photodisruption in the target tissue 1001. An applanation plate can be provided to be in contact with the target tissue 1001 to produce an interface for transmitting laser pulses to the target tissue 1001 and light coming from the target tissue 1001 through the interface. Notably, an optical imaging device 1030 is provided to capture light 1050 carrying target tissue images 1050 or imaging information from the target tissue 1001 to create an image of the target tissue 1001. The imaging signal 1032 from the imaging device 1030 is sent to a system control module 1040. The system control module 1040 operates to process the captured images from the image device 1030 and to control the optics module 1020 to adjust the position and focus of the surgical laser beam 1022 at the target tissue 1001 based on information from the captured images. The optics module 1020 can include one or more lenses and may further include one or more reflectors. A control actuator can be included in the optics module 1020 to adjust the focusing and the beam direction in response to a beam control signal 1044 from the system control module 1040. The control module 1040 can also control the pulsed laser 1010 via a laser control signal 1042.

The optical imaging device 1030 may be implemented to produce an optical imaging beam that is separate from the surgical laser beam 1022 to probe the target tissue 1001 and the returned light of the optical imaging beam is captured by the optical imaging device 1030 to obtain the images of the target tissue 1001. One example of such an optical imaging device 1030 is an optical coherence tomography (OCT) imaging module which uses two imaging beams, one probe beam directed to the target tissue 1001 thought the applanation plate and another reference beam in a reference optical path, to optically interfere with each other to obtain images of the target tissue 1001. In other implementations, the optical imaging device 1030 can use scattered or reflected light from the target tissue 1001 to capture images without sending a designated optical imaging beam to the target tissue 1001. For example, the imaging device 1030 can be a sensing array of sensing elements such as CCD or CMS sensors. For example, the images of photodisruption byproduct produced by the surgical laser beam 1022 may be captured by the optical imaging device 1030 for controlling the focusing and positioning of the surgical laser beam 1022. When the optical imaging device 1030 is designed to guide surgical laser beam alignment using the image of the photodisruption byproduct, the optical imaging device 1030 captures images of the photodisruption byproduct such as the laser-induced bubbles or cavities. The imaging device 1030 may also be an ultrasound imaging device to capture images based on acoustic images.

The system control module 1040 processes image data from the imaging device 1030 that includes the position offset information for the photodisruption byproduct from the target tissue position in the target tissue 1001. Based on the information obtained from the image, the beam control signal 1044 is generated to control the optics module 1020 which adjusts the laser beam 1022. A digital processing unit can be included in the system control module 1040 to perform various data processing for the laser alignment.

The above techniques and systems can be used deliver high repetition rate laser pulses to subsurface targets with a precision required for contiguous pulse placement, as needed for cutting or volume disruption applications. This can be accomplished with or without the use of a reference source on the surface of the target and can take into account movement of the target following applanation or during placement of laser pulses.

The applanation plate in the present systems is provided to facilitate and control precise, high speed positioning requirement for delivery of laser pulses into the tissue. Such an applanation plate can be made of a transparent material such as a glass with a predefined contact surface to the tissue so that the contact surface of the applanation plate forms a well-defined optical interface with the tissue. This well-defined interface can facilitate transmission and focusing of laser light into the tissue to control or reduce optical aberrations or variations (such as due to specific eye optical properties or changes that occur with surface drying) that are most critical at the air-tissue interface, which in the eye is at the anterior surface of the cornea. A number of contact lenses have been designed for various applications and targets inside the eye and other tissues, including ones that are disposable or reusable. The contact glass or applanation plate on the surface of the target tissue is used as a reference plate relative to which laser pulses are focused through the adjustment of focusing elements within the laser delivery system relative. Inherent in such an approach are the additional benefits afforded by the contact glass or applanation plate described previously, including control of the optical qualities of the tissue surface. Accordingly, laser pulses can be accurately placed at a high speed at a desired location (interaction point) in the target tissue relative to the applanation reference plate with little optical distortion of the laser pulses.

The optical imaging device 1030 in FIG. 7 captures images of the target tissue 1001 via the applanation plate. The control module 1040 processes the captured images to extract position information from the captured images and uses the extracted position information as a position reference or guide to control the position and focus of the surgical laser beam 1022. This imaging-guided laser surgery can be implemented without relying on the applanation plate as a position reference because the position of the applanation plate tends to change due to various factors as discussed above. Hence, although the applanation plate provides a desired optical interface for the surgical laser beam to enter the target tissue and to capture images of the target tissue, it may be difficult to use the applanation plate as a position reference to align and control the position and focus of the surgical laser beam for accurate delivery of laser pulses. The imaging-guided control of the position and focus of the surgical laser beam based on the imaging device 1030 and the control module 1040 allows the images of the target tissue 1001, e.g., images of inner structures of an eye, to be used as position references, without using the applanation plate to provide a position reference.

In addition to the physical effects of applanation that disproportionably affect the localization of internal tissue structures, in some surgical processes, it may be desirable for a targeting system to anticipate or account for nonlinear characteristics of photodisruption which can occur when using short pulse duration lasers. Photodisruption can cause complications in beam alignment and beam targeting. For example, one of the nonlinear optical effects in the tissue material when interacting with laser pulses during the photodisruption is that the refractive index of the tissue material experienced by the laser pulses is no longer a constant but varies with the intensity of the light. Because the intensity of the light in the laser pulses varies spatially within the pulsed laser beam, along and across the propagation direction of the pulsed laser beam, the refractive index of the tissue material also varies spatially. One consequence of this nonlinear refractive index is self-focusing or self-defocusing in the tissue material that changes the actual focus of and shifts the position of the focus of the pulsed laser beam inside the tissue. Therefore, a precise alignment of the pulsed laser beam to each target tissue position in the target tissue may also need to account for the nonlinear optical effects of the tissue material on the laser beam. The energy of the laser pulses may be adjusted to deliver the same physical effect in different regions of the target due to different physical characteristics, such as hardness, or due to optical considerations such as absorption or scattering of laser pulse light traveling to a particular region. In such cases, the differences in non-linear focusing effects between pulses of different energy values can also affect the laser alignment and laser targeting of the surgical pulses. In this regard, the direct images obtained from the target issue by the imaging device 1030 can be used to monitor the actual position of the surgical laser beam 1022 which reflects the combined effects of nonlinear optical effects in the target tissue and provide position references for control of the beam position and beam focus.

The techniques, apparatus and systems described here can be used in combination of an applanation plate to provide control of the surface shape and hydration, to reduce optical distortion, and provide for precise localization of photodisruption to internal structures through the applanated surface. The imaging-guided control of the beam position and focus described here can be applied to surgical systems and procedures that use means other than applanation plates to fix the eye, including the use of a suction ring which can lead to distortion or movement of the surgical target.

The following sections first describe examples of techniques, apparatus and systems for automated imaging-guided laser surgery based on varying degrees of integration of imaging functions into the laser control part of the systems. An optical or other modality imaging module, such as an OCT imaging module, can be used to direct a probe light or other type of beam to capture images of a target tissue, e.g., structures inside an eye. A surgical laser beam of laser pulses such as femtosecond or picosecond laser pulses can be guided by position information in the captured images to control the focusing and positioning of the surgical laser beam during the surgery. Both the surgical laser beam and the probe light beam can be sequentially or simultaneously directed to the target tissue during the surgery so that the surgical laser beam can be controlled based on the captured images to ensure precision and accuracy of the surgery.

Such imaging-guided laser surgery can be used to provide accurate and precise focusing and positioning of the surgical laser beam during the surgery because the beam control is based on images of the target tissue following applanation or fixation of the target tissue, either just before or nearly simultaneously with delivery of the surgical pulses. Notably, certain parameters of the target tissue such as the eye measured before the surgery may change during the surgery due to various factor such as preparation of the target tissue (e.g., fixating the eye to an applanation lens) and the alternation of the target tissue by the surgical operations. Therefore, measured parameters of the target tissue prior to such factors and/or the surgery may no longer reflect the physical conditions of the target tissue during the surgery. The present imaging-guided laser surgery can mitigate technical issues in connection with such changes for focusing and positioning the surgical laser beam before and during the surgery.

The present imaging-guided laser surgery may be effectively used for accurate surgical operations inside a target tissue. For example, when performing laser surgery inside the eye, laser light is focused inside the eye to achieve optical breakdown of the targeted tissue and such optical interactions can change the internal structure of the eye. For example, the crystalline lens can change its position, shape, thickness and diameter during accommodation, not only between prior measurement and surgery but also during surgery. Attaching the eye to the surgical instrument by mechanical means can change the shape of the eye in a not well defined way and further, the change can vary during surgery due to various factors, e.g., patient movement. Attaching means include fixating the eye with a suction ring and applanating the eye with a flat or curved lens. These changes amount to as much as a few millimeters. Mechanically referencing and fixating the surface of the eye such as the anterior surface of the cornea or limbus does not work well when performing precision laser microsurgery inside the eye.

The post preparation or near simultaneous imaging in the present imaging-guided laser surgery can be used to establish three-dimensional positional references between the inside features of the eye and the surgical instrument in an environment where changes occur prior to and during surgery. The positional reference information provided by the imaging prior to applanation and/or fixation of the eye, or during the actual surgery reflects the effects of changes in the eye and thus provides an accurate guidance to focusing and positioning of the surgical laser beam. A system based on the present imaging-guided laser surgery can be configured to be simple in structure and cost efficient. For example, a portion of the optical components associated with guiding the surgical laser beam can be shared with optical components for guiding the probe light beam for imaging the target tissue to simplify the device structure and the optical alignment and calibration of the imaging and surgical light beams.

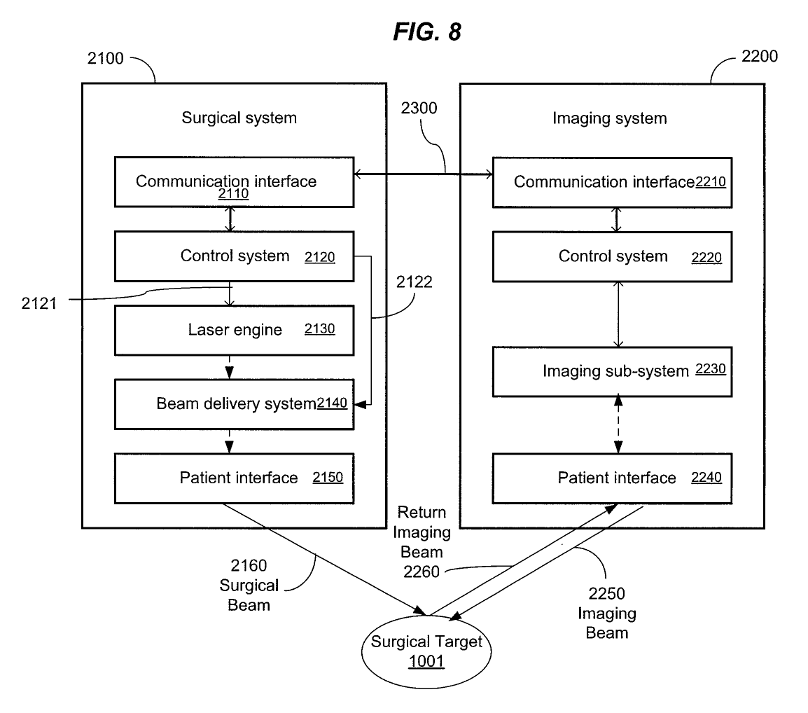

The imaging-guided laser surgical systems described below use the OCT imaging as an example of an imaging instrument and other non-OCT imaging devices may also be used to capture images for controlling the surgical lasers during the surgery. As illustrated in the examples below, integration of the imaging and surgical subsystems can be implemented to various degrees. In the simplest form without integrating hardware, the imaging and laser surgical subsystems are separated and can communicate to one another through interfaces. Such designs can provide flexibility in the designs of the two subsystems. Integration between the two subsystems, by some hardware components such as a patient interface, further expands the functionality by offering better registration of surgical area to the hardware components, more accurate calibration and may improve workflow. As the degree of integration between the two subsystems increases, such a system may be made increasingly cost-efficient and compact and system calibration will be further simplified and more stable over time. Examples for imaging-guided laser systems in FIGS. 8-16 are integrated at various degrees of integration.

One implementation of a present imaging-guided laser surgical system, for example, includes a surgical laser that produces a surgical laser beam of surgical laser pulses that cause surgical changes in a target tissue under surgery; a patient interface mount that engages a patient interface in contact with the target tissue to hold the target tissue in position; and a laser beam delivery module located between the surgical laser and the patient interface and configured to direct the surgical laser beam to the target tissue through the patient interface. This laser beam delivery module is operable to scan the surgical laser beam in the target tissue along a predetermined surgical pattern. This system also includes a laser control module that controls operation of the surgical laser and controls the laser beam delivery module to produce the predetermined surgical pattern and an OCT module positioned relative to the patient interface to have a known spatial relation with respect to the patient interface and the target issue fixed to the patient interface. The OCT module is configured to direct an optical probe beam to the target tissue and receive returned probe light of the optical probe beam from the target tissue to capture OCT images of the target tissue while the surgical laser beam is being directed to the target tissue to perform an surgical operation so that the optical probe beam and the surgical laser beam are simultaneously present in the target tissue. The OCT module is in communication with the laser control module to send information of the captured OCT images to the laser control module.

In addition, the laser control module in this particular system responds to the information of the captured OCT images to operate the laser beam delivery module in focusing and scanning of the surgical laser beam and adjusts the focusing and scanning of the surgical laser beam in the target tissue based on positioning information in the captured OCT images.

In some implementations, acquiring a complete image of a target tissue may not be necessary for registering the target to the surgical instrument and it may be sufficient to acquire a portion of the target tissue, e.g., a few points from the surgical region such as natural or artificial landmarks. For example, a rigid body has six degrees of freedom in 3D space and six independent points would be sufficient to define the rigid body. When the exact size of the surgical region is not known, additional points are needed to provide the positional reference. In this regard, several points can be used to determine the position and the curvature of the anterior and posterior surfaces, which are normally different, and the thickness and diameter of the crystalline lens of the human eye. Based on these data a body made up from two halves of ellipsoid bodies with given parameters can approximate and visualize a crystalline lens for practical purposes. In another implementation, information from the captured image may be combined with information from other sources, such as pre-operative measurements of lens thickness that are used as an input for the controller.

FIG. 8 shows one example of an imaging-guided laser surgical system with separated laser surgical system 2100 and imaging system 2200. The laser surgical system 2100 includes a laser engine 2130 with a surgical laser that produces a surgical laser beam 2160 of surgical laser pulses. A laser beam delivery module 2140 is provided to direct the surgical laser beam 2160 from the laser engine 2130 to the target tissue 1001 through a patient interface 2150 and is operable to scan the surgical laser beam 2160 in the target tissue 1001 along a predetermined surgical pattern. A laser control module 2120 is provided to control the operation of the surgical laser in the laser engine 2130 via a communication channel 2121 and controls the laser beam delivery module 2140 via a communication channel 2122 to produce the predetermined surgical pattern. A patient interface mount is provided to engage the patient interface 2150 in contact with the target tissue 1001 to hold the target tissue 1001 in position. The patient interface 2150 can be implemented to include a contact lens or applanation lens with a flat or curved surface to conformingly engage to the anterior surface of the eye and to hold the eye in position.

The imaging system 2200 in FIG. 8 can be an OCT module positioned relative to the patient interface 2150 of the surgical system 2100 to have a known spatial relation with respect to the patient interface 2150 and the target issue 1001 fixed to the patient interface 2150. This OCT module 2200 can be configured to have its own patient interface 2240 for interacting with the target tissue 1001. The imaging system 2200 includes an imaging control module 2220 and an imaging sub-system 2230. The sub-system 2230 includes a light source for generating imaging beam 2250 for imaging the target 1001 and an imaging beam delivery module to direct the optical probe beam or imaging beam 2250 to the target tissue 1001 and receive returned probe light 2260 of the optical imaging beam 2250 from the target tissue 1001 to capture OCT images of the target tissue 1001. Both the optical imaging beam 2250 and the surgical beam 2160 can be simultaneously directed to the target tissue 1001 to allow for sequential or simultaneous imaging and surgical operation.

As illustrated in FIG. 8, communication interfaces 2110 and 2210 are provided in both the laser surgical system 2100 and the imaging system 2200 to facilitate the communications between the laser control by the laser control module 2120 and imaging by the imaging system 2200 so that the OCT module 2200 can send information of the captured OCT images to the laser control module 2120. The laser control module 2120 in this system responds to the information of the captured OCT images to operate the laser beam delivery module 2140 in focusing and scanning of the surgical laser beam 2160 and dynamically adjusts the focusing and scanning of the surgical laser beam 2160 in the target tissue 1001 based on positioning information in the captured OCT images. The integration between the laser surgical system 2100 and the imaging system 2200 is mainly through communication between the communication interfaces 2110 and 2210 at the software level.

In this and other examples, various subsystems or devices may also be integrated. For example, certain diagnostic instruments such as wavefront aberrometers, corneal topography measuring devices may be provided in the system, or pre-operative information from these devices can be utilized to augment intra-operative imaging.

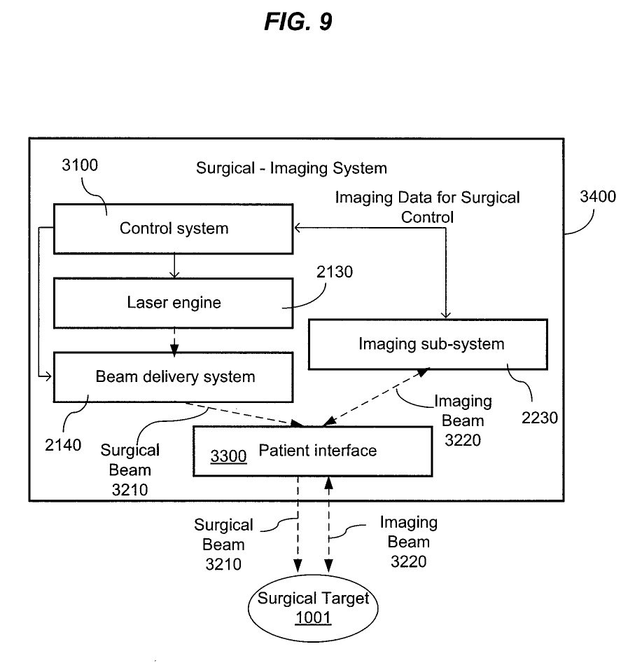

FIG. 9 shows an example of an imaging-guided laser surgical system with additional integration features. The imaging and surgical systems share a common patient interface 3300 which immobilizes target tissue 1001 (e.g., the eye) without having two separate patient interfaces as in FIG. 8. The surgical beam 3210 and the imaging beam 3220 are combined at the patient interface 3330 and are directed to the target 1001 by the common patient interface 3300. In addition, a common control module 3100 is provided to control both the imaging sub-system 2230 and the surgical part (the laser engine 2130 and the beam delivery system 2140). This increased integration between imaging and surgical parts allows accurate calibration of the two subsystems and the stability of the position of the patient and surgical volume. A common housing 3400 is provided to enclose both the surgical and imaging subsystems. When the two systems are not integrated into a common housing, the common patient interface 3300 can be part of either the imaging or the surgical subsystem.

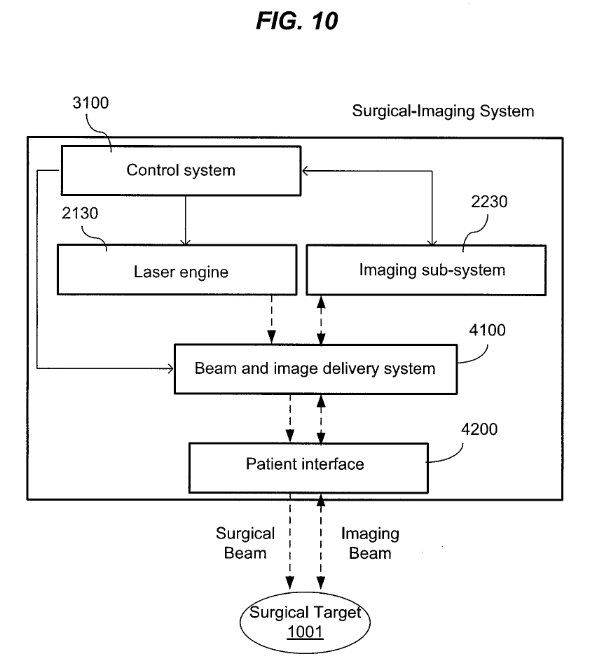

FIG. 10 shows an example of an imaging-guided laser surgical system where the laser surgical system and the imaging system share both a common beam delivery module 4100 and a common patient interface 4200. This integration further simplifies the system structure and system control operation.

In one implementation, the imaging system in the above and other examples can be an optical computed tomography (OCT) system and the laser surgical system is a femtosecond or picosecond laser based ophthalmic surgical system. In OCT, light from a low coherence, broadband light source such as a super luminescent diode is split into separate reference and signal beams. The signal beam is the imaging beam sent to the surgical target and the returned light of the imaging beam is collected and recombined coherently with the reference beam to form an interferometer. Scanning the signal beam perpendicularly to the optical axis of the optical train or the propagation direction of the light provides spatial resolution in the x-y direction while depth resolution comes from extracting differences between the path lengths of the reference arm and the returned signal beam in the signal arm of the interferometer. While the x-y scanner of different OCT implementations are essentially the same, comparing the path lengths and getting z-scan information can happen in different ways. In one implementation known as the time domain OCT, for example, the reference arm is continuously varied to change its path length while a photodetector detects interference modulation in the intensity of the re-combined beam. In a different implementation, the reference arm is essentially static and the spectrum of the combined light is analyzed for interference. The Fourier transform of the spectrum of the combined beam provides spatial information on the scattering from the interior of the sample. This method is known as the spectral domain or Fourier OCT method. In a different implementation known as a frequency swept OCT (S. R. Chinn, et. al., Opt. Lett. 22, 1997), a narrowband light source is used with its frequency swept rapidly across a spectral range. Interference between the reference and signal arms is detected by a fast detector and dynamic signal analyzer. An external cavity tuned diode laser or frequency tuned of frequency domain mode-locked (FDML) laser developed for this purpose (R. Huber et. Al. Opt. Express, 13, 2005) (S. H. Yun, IEEE J. of Sel. Q. El. 3(4) p. 1087-1096, 1997) can be used in these examples as a light source. A femtosecond laser used as a light source in an OCT system can have sufficient bandwidth and can provide additional benefits of increased signal to noise ratios.

The OCT imaging device in the systems in this document can be used to perform various imaging functions. For example, the OCT can be used to suppress complex conjugates resulting from the optical configuration of the system or the presence of the applanation plate, capture OCT images of selected locations inside the target tissue to provide three-dimensional positioning information for controlling focusing and scanning of the surgical laser beam inside the target tissue, or capture OCT images of selected locations on the surface of the target tissue or on the applanation plate to provide positioning registration for controlling changes in orientation that occur with positional changes of the target, such as from upright to supine. The OCT can be calibrated by a positioning registration process based on placement of marks or markers in one positional orientation of the target that can then be detected by the OCT module when the target is in another positional orientation. In other implementations, the OCT imaging system can be used to produce a probe light beam that is polarized to optically gather the information on the internal structure of the eye. The laser beam and the probe light beam may be polarized in different polarizations. The OCT can include a polarization control mechanism that controls the probe light used for said optical tomography to polarize in one polarization when traveling toward the eye and in a different polarization when traveling away from the eye. The polarization control mechanism can include, e.g., a wave-plate or a Faraday rotator.

The system in FIG. 10 is shown as a spectral OCT configuration and can be configured to share the focusing optics part of the beam delivery module between the surgical and the imaging systems. The main requirements for the optics are related to the operating wavelength, image quality, resolution, distortion etc. The laser surgical system can be a femtosecond laser system with a high numerical aperture system designed to achieve diffraction limited focal spot sizes, e.g., about 2 to 3 micrometers. Various femtosecond ophthalmic surgical lasers can operate at various wavelengths such as wavelengths of around 1.05 micrometer. The operating wavelength of the imaging device can be selected to be close to the laser wavelength so that the optics is chromatically compensated for both wavelengths. Such a system may include a third optical channel, a visual observation channel such as a surgical microscope, to provide an additional imaging device to capture images of the target tissue. If the optical path for this third optical channel shares optics with the surgical laser beam and the light of the OCT imaging device, the shared optics can be configured with chromatic compensation in the visible spectral band for the third optical channel and the spectral bands for the surgical laser beam and the OCT imaging beam.

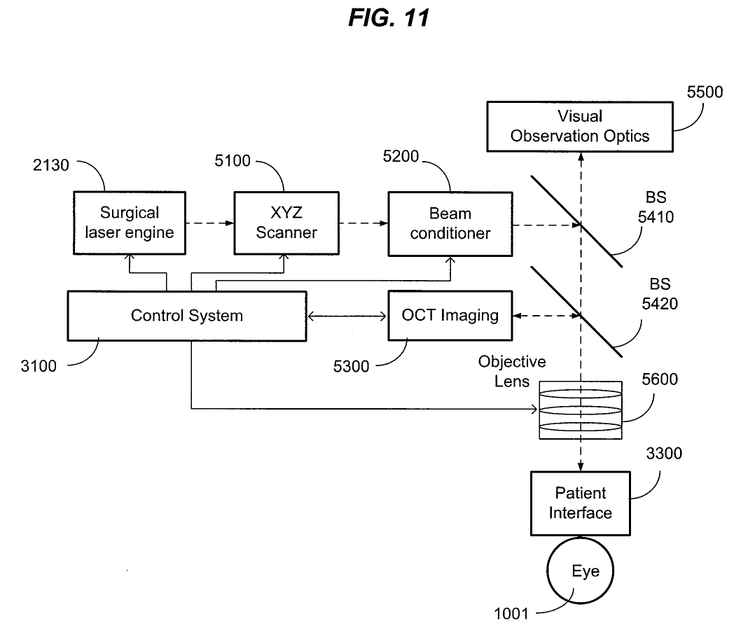

FIG. 11 shows a particular example of the design in FIG. 9 where the scanner 5100 for scanning the surgical laser beam and the beam conditioner 5200 for conditioning (collimating and focusing) the surgical laser beam are separate from the optics in the OCT imaging module 5300 for controlling the imaging beam for the OCT. The surgical and imaging systems share an objective lens 5600 module and the patient interface 3300. The objective lens 5600 directs and focuses both the surgical laser beam and the imaging beam to the patient interface 3300 and its focusing is controlled by the control module 3100. Two beam splitters 5410 and 5420 are provided to direct the surgical and imaging beams. The beam splitter 5420 is also used to direct the returned imaging beam back into the OCT imaging module 5300. Two beam splitters 5410 and 5420 also direct light from the target 1001 to a visual observation optics unit 5500 to provide direct view or image of the target 1001. The unit 5500 can be a lens imaging system for the surgeon to view the target 1001 or a camera to capture the image or video of the target 1001. Various beam splitters can be used, such as dichroic and polarization beam splitters, optical grating, holographic beam splitter or a combinations of these.

In some implementations, the optical components may be appropriately coated with antireflection coating for both the surgical and for the OCT wavelength to reduce glare from multiple surfaces of the optical beam path. Reflections would otherwise reduce the throughput of the system and reduce the signal to noise ratio by increasing background light in the OCT imaging unit. One way to reduce glare in the OCT is to rotate the polarization of the return light from the sample by wave-plate of Faraday isolator placed close to the target tissue and orient a polarizer in front of the OCT detector to preferentially detect light returned from the sample and suppress light scattered from the optical components.

In a laser surgical system, each of the surgical laser and the OCT system can have a beam scanner to cover the same surgical region in the target tissue. Hence, the beam scanning for the surgical laser beam and the beam scanning for the imaging beam can be integrated to share common scanning devices.

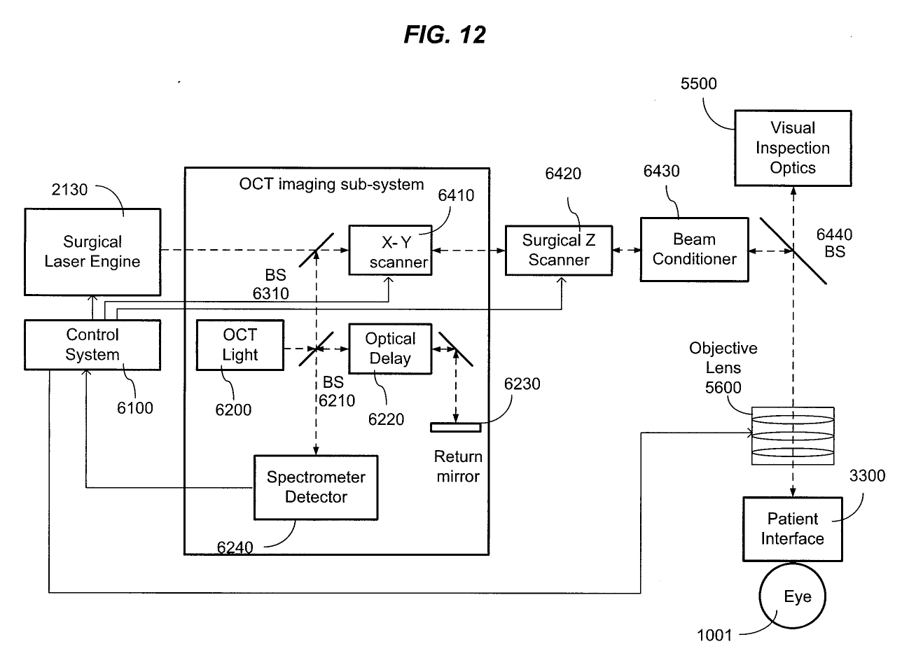

FIG. 12 shows an example of such a system in detail. In this implementation the x-y scanner 6410 and the z scanner 6420 are shared by both subsystems. A common control 6100 is provided to control the system operations for both surgical and imaging operations. The OCT sub-system includes an OCT light source 6200 that produce the imaging light that is split into an imaging beam and a reference beam by a beam splitter 6210. The imaging beam is combined with the surgical beam at the beam splitter 6310 to propagate along a common optical path leading to the target 1001. The scanners 6410 and 6420 and the beam conditioner unit 6430 are located downstream from the beam splitter 6310. A beam splitter 6440 is used to direct the imaging and surgical beams to the objective lens 5600 and the patient interface 3300.

In the OCT sub-system, the reference beam transmits through the beam splitter 6210 to an optical delay device 6220 and is reflected by a return mirror 6230. The returned imaging beam from the target 1001 is directed back to the beam splitter 6310 which reflects at least a portion of the returned imaging beam to the beam splitter 6210 where the reflected reference beam and the returned imaging beam overlap and interfere with each other. A spectrometer detector 6240 is used to detect the interference and to produce OCT images of the target 1001. The OCT image information is sent to the control system 6100 for controlling the surgical laser engine 2130, the scanners 6410 and 6420 and the objective lens 5600 to control the surgical laser beam. In one implementation, the optical delay device 6220 can be varied to change the optical delay to detect various depths in the target tissue 1001.

If the OCT system is a time domain system, the two subsystems use two different z-scanners because the two scanners operate in different ways. In this example, the z scanner of the surgical system operates by changing the divergence of the surgical beam in the beam conditioner unit without changing the path lengths of the beam in the surgical beam path. On the other hand, the time domain OCT scans the z-direction by physically changing the beam path by a variable delay or by moving the position of the reference beam return mirror. After calibration, the two z-scanners can be synchronized by the laser control module. The relationship between the two movements can be simplified to a linear or polynomial dependence, which the control module can handle or alternatively calibration points can define a look-up table to provide proper scaling. Spectral/Fourier domain and frequency swept source OCT devices have no z-scanner, the length of the reference arm is static. Besides reducing costs, cross calibration of the two systems will be relatively straightforward. There is no need to compensate for differences arising from image distortions in the focusing optics or from the differences of the scanners of the two systems since they are shared.

In practical implementations of the surgical systems, the focusing objective lens 5600 is slidably or movably mounted on a base and the weight of the objective lens is balanced to limit the force on the patient's eye. The patient interface 3300 can include an applanation lens attached to a patient interface mount. The patient interface mount is attached to a mounting unit, which holds the focusing objective lens. This mounting unit is designed to ensure a stable connection between the patient interface and the system in case of unavoidable movement of the patient and allows gentler docking of the patient interface onto the eye. Various implementations for the focusing objective lens can be used and one example is described in U.S. Pat. No. 5,336,215 to Hsueh. This presence of an adjustable focusing objective lens can change the optical path length of the optical probe light as part of the optical interferometer for the OCT sub-system. Movement of the objective lens 5600 and patient interface 3300 can change the path length differences between the reference beam and the imaging signal beam of the OCT in an uncontrolled way and this may degrade the OCT depth information detected by the OCT. This would happen not only in time-domain but also in spectral/Fourier domain and frequency-swept OCT systems.

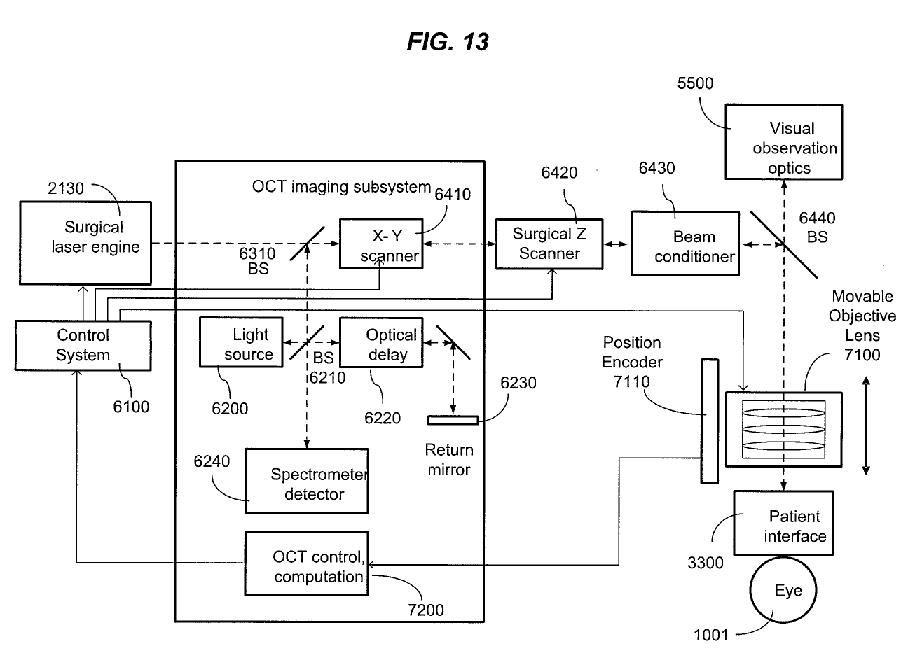

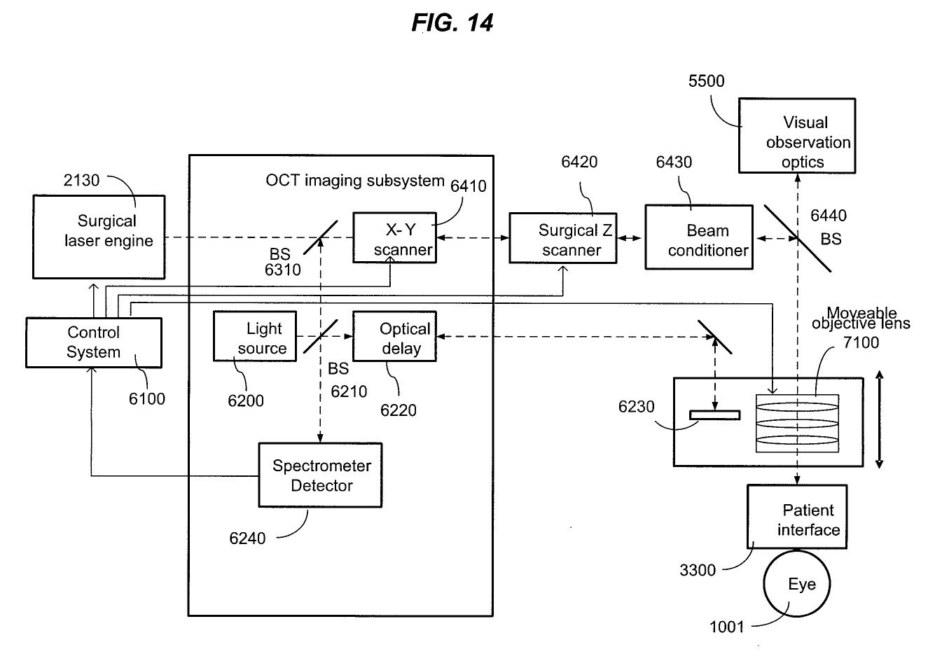

FIGS. 13-14 show exemplary imaging-guided laser surgical systems that address the technical issue associated with the adjustable focusing objective lens.

The system in FIG. 13 provides a position sensing device 7110 coupled to the movable focusing objective lens 7100 to measure the position of the objective lens 7100 on a slideable mount and communicates the measured position to a control module 7200 in the OCT system. The control system 6100 can control and move the position of the objective lens 7100 to adjust the optical path length traveled by the imaging signal beam for the OCT operation and the position of the lens 7100 is measured and monitored by the position encoder 7110 and direct fed to the OCT control 7200. The control module 7200 in the OCT system applies an algorithm, when assembling a 3D image in processing the OCT data, to compensate for differences between the reference arm and the signal arm of the interferometer inside the OCT caused by the movement of the focusing objective lens 7100 relative to the patient interface 3300. The proper amount of the change in the position of the lens 7100 computed by the OCT control module 7200 is sent to the control 6100 which controls the lens 7100 to change its position.

FIG. 14 shows another exemplary system where the return mirror 6230 in the reference arm of the interferometer of the OCT system or at least one part in an optical path length delay assembly of the OCT system is rigidly attached to the movable focusing objective lens 7100 so the signal arm and the reference arm undergo the same amount of change in the optical path length when the objective lens 7100 moves. As such, the movement of the objective lens 7100 on the slide is automatically compensated for path-length differences in the OCT system without additional need for a computational compensation.

The above examples for imaging-guided laser surgical systems, the laser surgical system and the OCT system use different light sources. In an even more complete integration between the laser surgical system and the OCT system, a femtosecond surgical laser as a light source for the surgical laser beam can also be used as the light source for the OCT system.

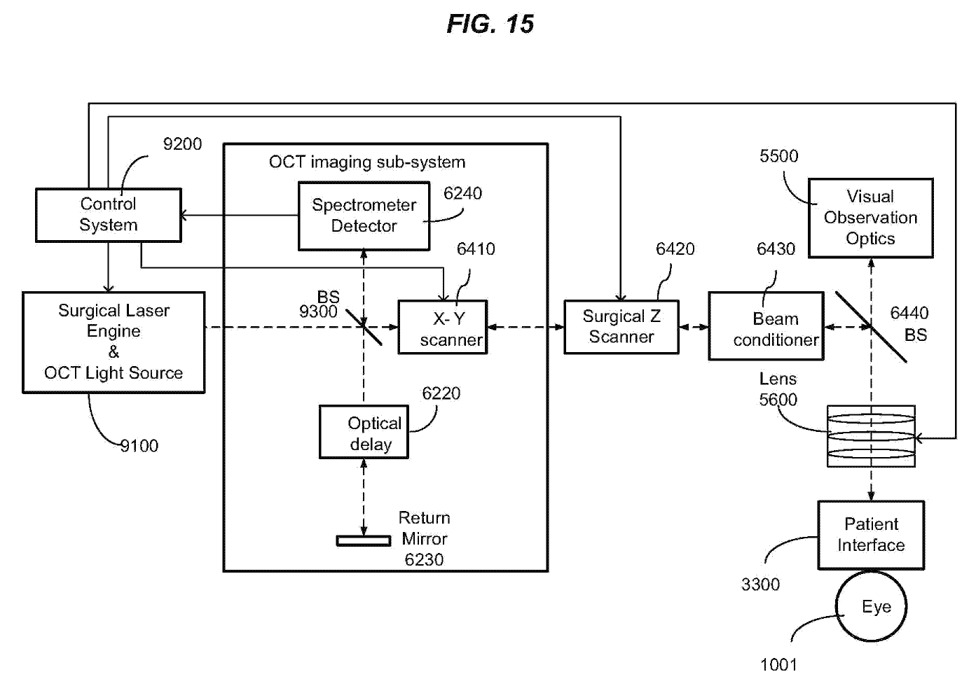

FIG. 15 shows an example where a femtosecond pulse laser in a light module 9100 is used to generate both the surgical laser beam for surgical operations and the probe light beam for OCT imaging. A beam splitter 9300 is provided to split the laser beam into a first beam as both the surgical laser beam and the signal beam for the OCT and a second beam as the reference beam for the OCT. The first beam is directed through an x-y scanner 6410 which scans the beam in the x and y directions perpendicular to the propagation direction of the first beam and a second scanner (z scanner) 6420 that changes the divergence of the beam to adjust the focusing of the first beam at the target tissue 1001. This first beam performs the surgical operations at the target tissue 1001 and a portion of this first beam is back scattered to the patient interface and is collected by the objective lens as the signal beam for the signal arm of the optical interferometer of the OCT system. This returned light is combined with the second beam that is reflected by a return mirror 6230 in the reference arm and is delayed by an adjustable optical delay element 6220 for a time-domain OCT to control the path difference between the signal and reference beams in imaging different depths of the target tissue 1001. The control system 9200 controls the system operations.

Surgical practice on the cornea has shown that a pulse duration of several hundred femtoseconds may be sufficient to achieve good surgical performance, while for OCT of a sufficient depth resolution broader spectral bandwidth generated by shorter pulses, e.g., below several tens of femtoseconds, are needed. In this context, the design of the OCT device dictates the duration of the pulses from the femtosecond surgical laser.

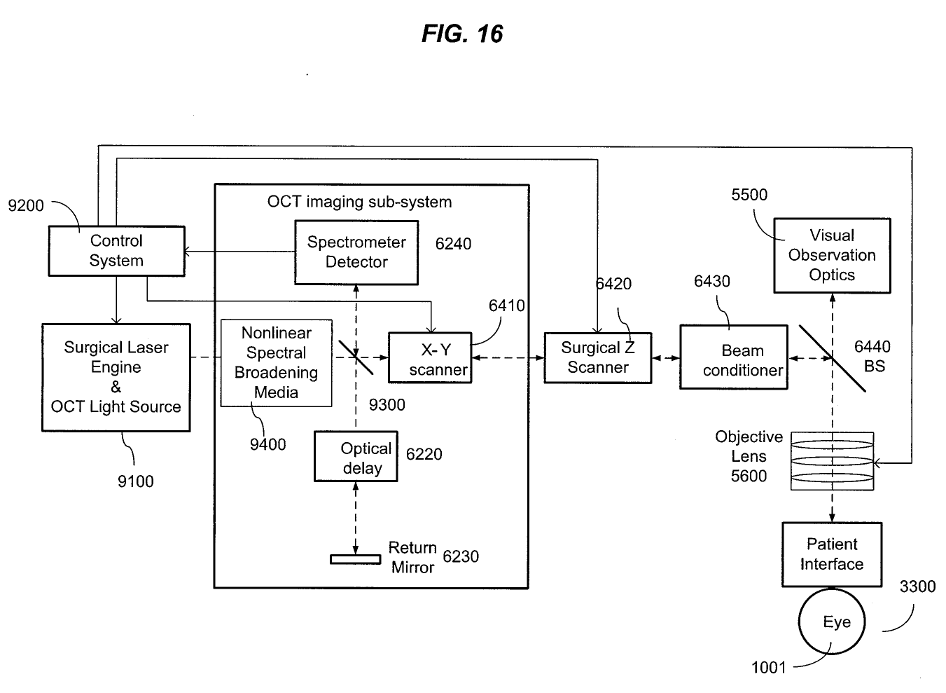

FIG. 16 shows another imaging-guided system that uses a single pulsed laser 9100 to produce the surgical light and the imaging light. A nonlinear spectral broadening media 9400 is placed in the output optical path of the femtosecond pulsed laser to use an optical non-linear process such as white light generation or spectral broadening to broaden the spectral bandwidth of the pulses from a laser source of relatively longer pulses, several hundred femtoseconds normally used in surgery. The media 9400 can be a fiber-optic material, for example. The light intensity requirements of the two systems are different and a mechanism to adjust beam intensities can be implemented to meet such requirements in the two systems. For example, beam steering mirrors, beam shutters or attenuators can be provided in the optical paths of the two systems to properly control the presence and intensity of the beam when taking an OCT image or performing surgery in order to protect the patient and sensitive instruments from excessive light intensity.

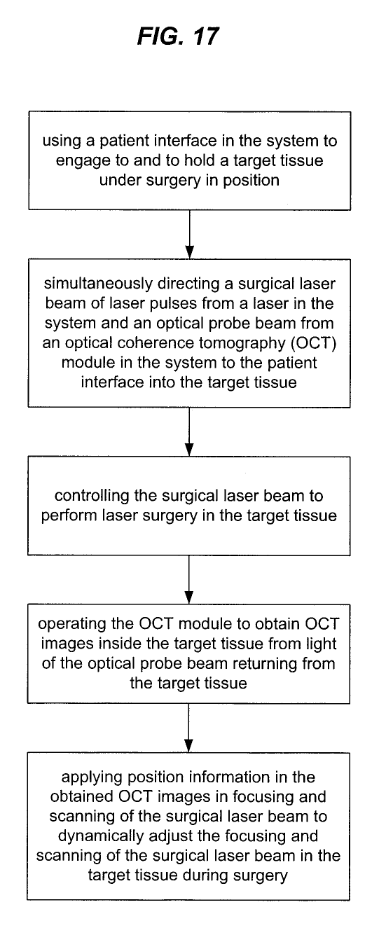

In operation, the above examples in FIGS. 8-16 can be used to perform imaging-guided laser surgery. FIG. 17 shows one example of a method for performing laser surgery by using an imaging-guided laser surgical system. This method uses a patient interface in the system to engage to and to hold a target tissue under surgery in position and simultaneously directs a surgical laser beam of laser pulses from a laser in the system and an optical probe beam from the OCT module in the system to the patient interface into the target tissue. The surgical laser beam is controlled to perform laser surgery in the target tissue and the OCT module is operated to obtain OCT images inside the target tissue from light of the optical probe beam returning from the target tissue. The position information in the obtained OCT images is applied in focusing and scanning of the surgical laser beam to adjust the focusing and scanning of the surgical laser beam in the target tissue before or during surgery.

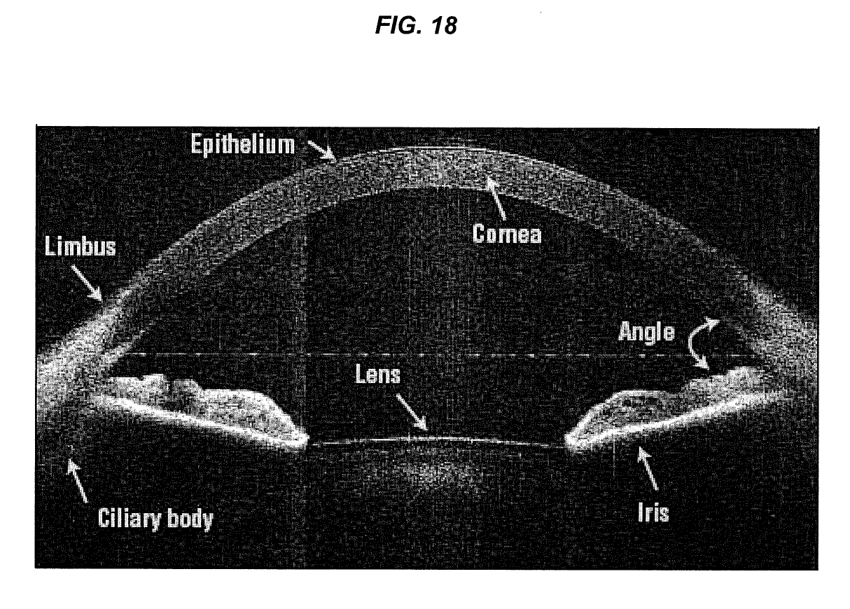

FIG. 18 shows an example of an OCT image of an eye. The contacting surface of the applanation lens in the patent interface can be configured to have a curvature that minimizes distortions or folds in the cornea due to the pressure exerted on the eye during applanation. After the eye is successfully applanated at the patient interface, an OCT image can be obtained. As illustrated in FIG. 18, the curvature of the lens and cornea as well as the distances between the lens and cornea are identifiable in the OCT image. Subtler features such as the epithelium-cornea interface are detectable. Each of these identifiable features may be used as an internal reference of the laser coordinates with the eye. The coordinates of the cornea and lens can be digitized using well-established computer vision algorithms such as Edge or Blob detection. Once the coordinates of the lens are established, they can be used to control the focusing and positioning of the surgical laser beam for the surgery.

Alternatively, a calibration sample material may be used to form a 3-D array of reference marks at locations with known position coordinates. The OCT image of the calibration sample material can be obtained to establish a mapping relationship between the known position coordinates of the reference marks and the OCT images of the reference marks in the obtained OCT image. This mapping relationship is stored as digital calibration data and is applied in controlling the focusing and scanning of the surgical laser beam during the surgery in the target tissue based on the OCT images of the target tissue obtained during the surgery. The OCT imaging system is used here as an example and this calibration can be applied to images obtained via other imaging techniques.

In an imaging-guided laser surgical system described here, the surgical laser can produce relatively high peak powers sufficient to drive strong field/multi-photon ionization inside of the eye (i.e. inside of the cornea and lens) under high numerical aperture focusing. Under these conditions, one pulse from the surgical laser generates a plasma within the focal volume. Cooling of the plasma results in a well defined damage zone or “bubble” that may be used as a reference point. The following sections describe a calibration procedure for calibrating the surgical laser against an OCT-based imaging system using the damage zones created by the surgical laser.

Before surgery can be performed, the OCT is calibrated against the surgical laser to establish a relative positioning relationship so that the surgical laser can be controlled in position at the target tissue with respect to the position associated with images in the OCT image of the target tissue obtained by the OCT. One way for performing this calibration uses a pre-calibrated target or “phantom” which can be damaged by the laser as well as imaged with the OCT. The phantom can be fabricated from various materials such as a glass or hard plastic (e.g. PMMA) such that the material can permanently record optical damage created by the surgical laser. The phantom can also be selected to have optical or other properties (such as water content) that are similar to the surgical target.

The phantom can be, e.g., a cylindrical material having a diameter of at least 10 mm (or that of the scanning range of the delivery system) and a cylindrical length of at least 10 mm long spanning the distance of the epithelium to the crystalline lens of the eye, or as long as the scanning depth of the surgical system. The upper surface of the phantom can be curved to mate seamlessly with the patient interface or the phantom material may be compressible to allow full applanation. The phantom may have a three dimensional grid such that both the laser position (in x and y) and focus (z), as well as the OCT image can be referenced against the phantom.

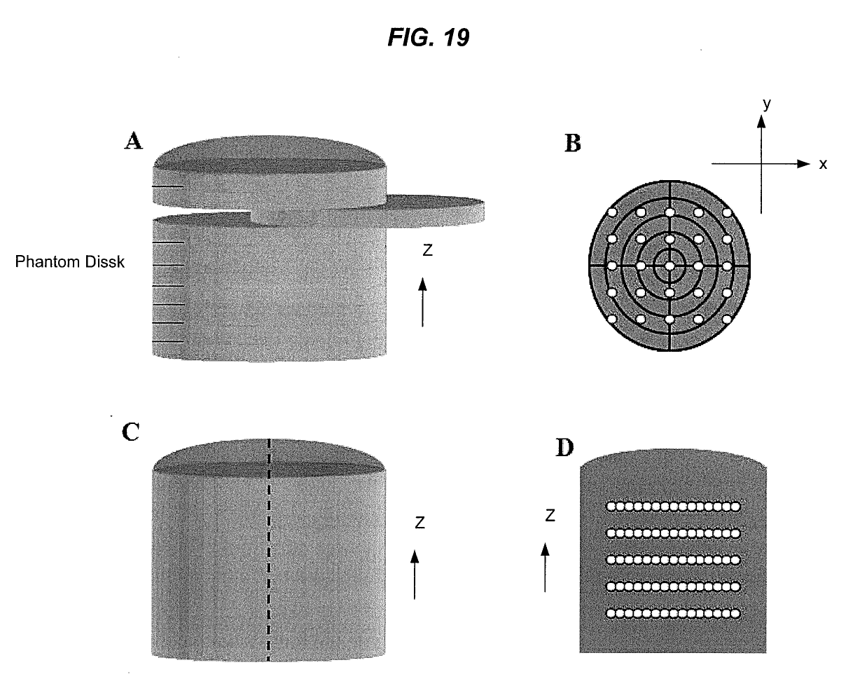

FIGS. 19A-19D illustrate two exemplary configurations for the phantom. FIG. 19A illustrates a phantom that is segmented into thin disks. FIG. 19B shows a single disk patterned to have a grid of reference marks as a reference for determining the laser position across the phantom (i.e. the x- and y-coordinates). The z-coordinate (depth) can be determined by removing an individual disk from the stack and imaging it under a confocal microscope.

FIG. 19C illustrates a phantom that can be separated into two halves. Similar to the segmented phantom in FIG. 19A, this phantom is structured to contain a grid of reference marks as a reference for determining the laser position in the x- and y-coordinates. Depth information can be extracted by separating the phantom into the two halves and measuring the distance between damage zones. The combined information can provide the parameters for image guided surgery.

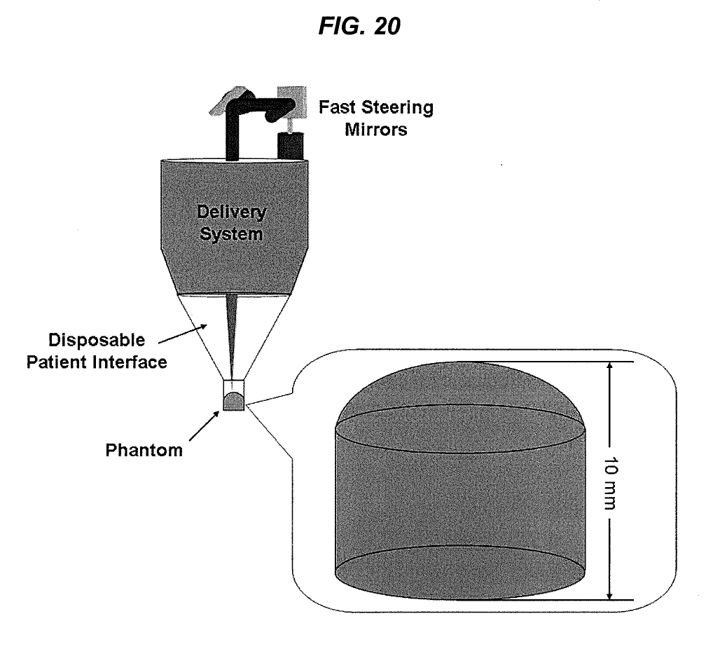

FIG. 20 shows a surgical system part of the imaging-guided laser surgical system. This system includes steering mirrors which may be actuated by actuators such as galvanometers or voice coils, an objective lens e and a disposable patient interface. The surgical laser beam is reflected from the steering mirrors through the objective lens. The objective lens focuses the beam just after the patient interface. Scanning in the x- and y-coordinates is performed by changing the angle of the beam relative to the objective lens. Scanning in z-plane is accomplished by changing the divergence of the incoming beam using a system of lens upstream to the steering mirrors.

In this example, the conical section of the disposable patient interface may be either air spaced or solid and the section interfacing with the patient includes a curved contact lens. The curved contact lens can be fabricated from fused silica or other material resistant to forming color centers when irradiated with ionizing radiation. The radius of curvature is on the upper limit of what is compatible with the eye, e.g., about 10 mm.

The first step in the calibration procedure is docking the patient interface with the phantom. The curvature of the phantom matches the curvature of the patient interface. After docking, the next step in the procedure involves creating optical damage inside of the phantom to produce the reference marks.

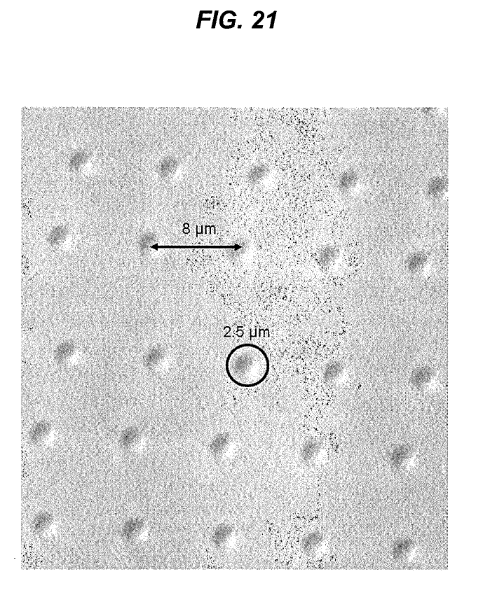

FIG. 21 shows examples of actual damage zones produced by a femtosecond laser in glass. The separation between the damage zones is on average 8 μm (the pulse energy is 2.2 μJ with duration of 580 fs at full width at half maximum). The optical damage depicted in FIG. 21 shows that the damage zones created by the femtosecond laser are well-defined and discrete. In the example shown, the damage zones have a diameter of about 2.5 μm. Optical damage zones similar to that shown in FIG. 20 are created in the phantom at various depths to form a 3-D array of the reference marks. These damage zones are referenced against the calibrated phantom either by extracting the appropriate disks and imaging it under a confocal microscope (FIG. 19A) or by splitting the phantom into two halves and measuring the depth using a micrometer (FIG. 19C). The x- and y-coordinates can be established from the pre-calibrated grid.

After damaging the phantom with the surgical laser, OCT on the phantom is performed. The OCT imaging system provides a 3D rendering of the phantom establishing a relationship between the OCT coordinate system and the phantom. The damage zones are detectable with the imaging system. The OCT and laser may be cross-calibrated using the phantom's internal standard. After the OCT and the laser are referenced against each other, the phantom can be discarded.

Prior to surgery, the calibration can be verified. This verification step involves creating optical damage at various positions inside of a second phantom. The optical damage should be intense enough such that the multiple damage zones which create a circular pattern can be imaged by the OCT. After the pattern is created, the second phantom is imaged with the OCT. Comparison of the OCT image with the laser coordinates provides the final check of the system calibration prior to surgery.

Once the coordinates are fed into the laser, laser surgery can be performed inside the eye. This involves photo-emulsification of the lens using the laser, as well as other laser treatments to the eye. The surgery can be stopped at any time and the anterior segment of the eye (FIG. 17) can be re-imaged to monitor the progress of the surgery; moreover, after the IOL is inserted, imaging the IOL (with light or no applanation) provides information regarding the position of the IOL in the eye. This information may be utilized by the physician to refine the position of the IOL.

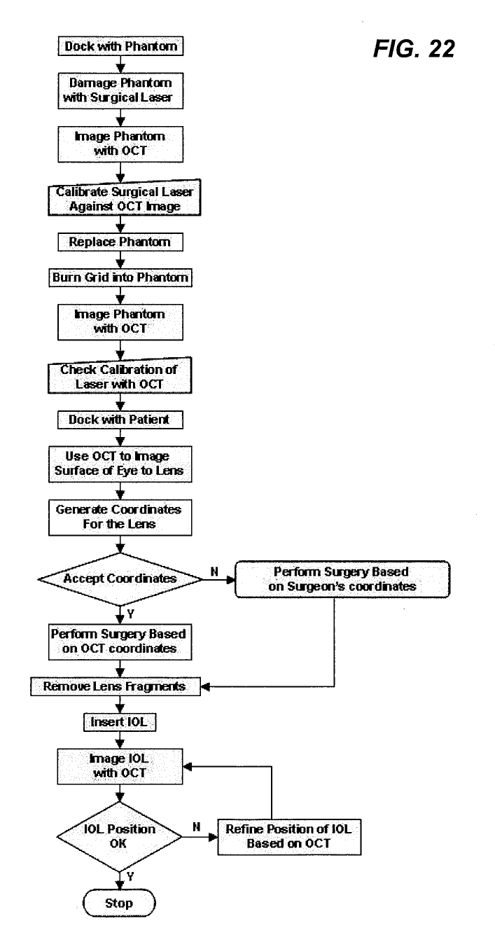

FIG. 22 shows an example of the calibration process and the post-calibration surgical operation. This examples illustrates a method for performing laser surgery by using an imaging-guided laser surgical system can include using a patient interface in the system, that is engaged to hold a target tissue under surgery in position, to hold a calibration sample material during a calibration process before performing a surgery; directing a surgical laser beam of laser pulses from a laser in the system to the patient interface into the calibration sample material to burn reference marks at selected three-dimensional reference locations; directing an optical probe beam from an optical coherence tomography (OCT) module in the system to the patient interface into the calibration sample material to capture OCT images of the burnt reference marks; and establishing a relationship between positioning coordinates of the OCT module and the burnt reference marks. After the establishing the relationship, a patient interface in the system is used to engage to and to hold a target tissue under surgery in position. The surgical laser beam of laser pulses and the optical probe beam are directed to the patient interface into the target tissue. The surgical laser beam is controlled to perform laser surgery in the target tissue. The OCT module is operated to obtain OCT images inside the target tissue from light of the optical probe beam returning from the target tissue and the position information in the obtained OCT images and the established relationship are applied in focusing and scanning of the surgical laser beam to adjust the focusing and scanning of the surgical laser beam in the target tissue during surgery. While such calibrations can be performed immediately prior to laser surgery, they can also be performed at various intervals before a procedure, using calibration validations that demonstrated a lack of drift or change in calibration during such intervals.

The following examples describe imaging-guided laser surgical techniques and systems that use images of laser-induced photodisruption byproducts for alignment of the surgical laser beam.

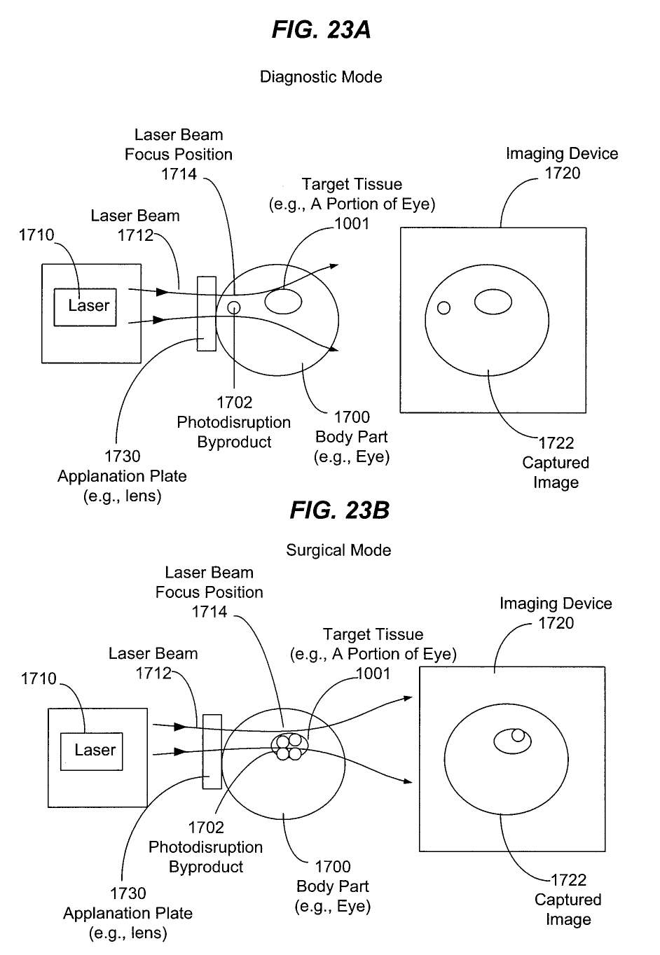

FIGS. 23A-B illustrate another implementation of the present technique in which actual photodisruption byproducts in the target tissue are used to guide further laser placement. A pulsed laser 1710, such as a femtosecond or picosecond laser, is used to produce a laser beam 1712 with laser pulses to cause photodisruption in a target tissue 1001. The target tissue 1001 may be a part of a body part 1700 of a subject, e.g., a portion of the lens of one eye. The laser beam 1712 is focused and directed by an optics module for the laser 1710 to a target tissue position in the target tissue 1001 to achieve a certain surgical effect. The target surface is optically coupled to the laser optics module by an applanation plate 1730 that transmits the laser wavelength, as well as image wavelengths from the target tissue. The applanation plate 1730 can be an applanation lens. An imaging device 1720 is provided to collect reflected or scattered light or sound from the target tissue 1001 to capture images of the target tissue 1001 either before or after (or both) the applanation plate is applied. The captured imaging data is then processed by the laser system control module to determine the desired target tissue position. The laser system control module moves or adjusts optical or laser elements based on standard optical models to ensure that the center of photodisruption byproduct 1702 overlaps with the target tissue position. This can be a dynamic alignment process where the images of the photodisruption byproduct 1702 and the target tissue 1001 are continuously monitored during the surgical process to ensure that the laser beam is properly positioned at each target tissue position.

In one implementation, the laser system can be operated in two modes: first in a diagnostic mode in which the laser beam 1712 is initially aligned by using alignment laser pulses to create photodisruption byproduct 1702 for alignment and then in a surgical mode where surgical laser pulses are generated to perform the actual surgical operation. In both modes, the images of the disruption byproduct 1702 and the target tissue 1001 are monitored to control the beam alignment. FIG. 17A shows the diagnostic mode where the alignment laser pulses in the laser beam 1712 may be set at a different energy level than the energy level of the surgical laser pulses. For example, the alignment laser pulses may be less energetic than the surgical laser pulses but sufficient to cause significant photodisruption in the tissue to capture the photodisruption byproduct 1702 at the imaging device 1720. The resolution of this coarse targeting may not be sufficient to provide desired surgical effect. Based on the captured images, the laser beam 1712 can be aligned properly. After this initial alignment, the laser 1710 can be controlled to produce the surgical laser pulses at a higher energy level to perform the surgery. Because the surgical laser pulses are at a different energy level than the alignment laser pulses, the nonlinear effects in the tissue material in the photodisruption can cause the laser beam 1712 to be focused at a different position from the beam position during the diagnostic mode. Therefore, the alignment achieved during the diagnostic mode is a coarse alignment and additional alignment can be further performed to precisely position each surgical laser pulse during the surgical mode when the surgical laser pulses perform the actual surgery. Referring to FIG. 23A, the imaging device 1720 captures the images from the target tissue 1001 during the surgical mode and the laser control module adjust the laser beam 1712 to place the focus position 1714 of the laser beam 1712 onto the desired target tissue position in the target tissue 1001. This process is performed for each target tissue position.

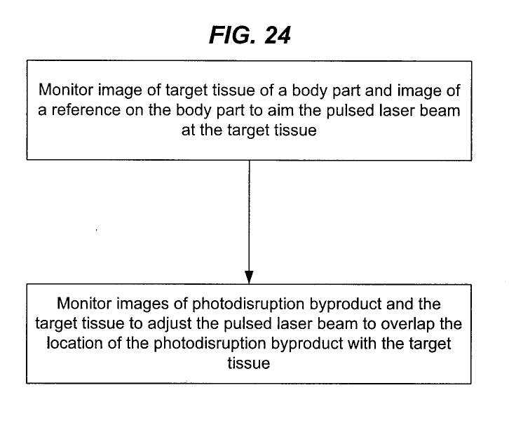

FIG. 24 shows one implementation of the laser alignment where the laser beam is first approximately aimed at the target tissue and then the image of the photodisruption byproduct is captured and used to align the laser beam. The image of the target tissue of the body part as the target tissue and the image of a reference on the body part are monitored to aim the pulsed laser beam at the target tissue. The images of photodisruption byproduct and the target tissue are used to adjust the pulsed laser beam to overlap the location of the photodisruption byproduct with the target tissue.

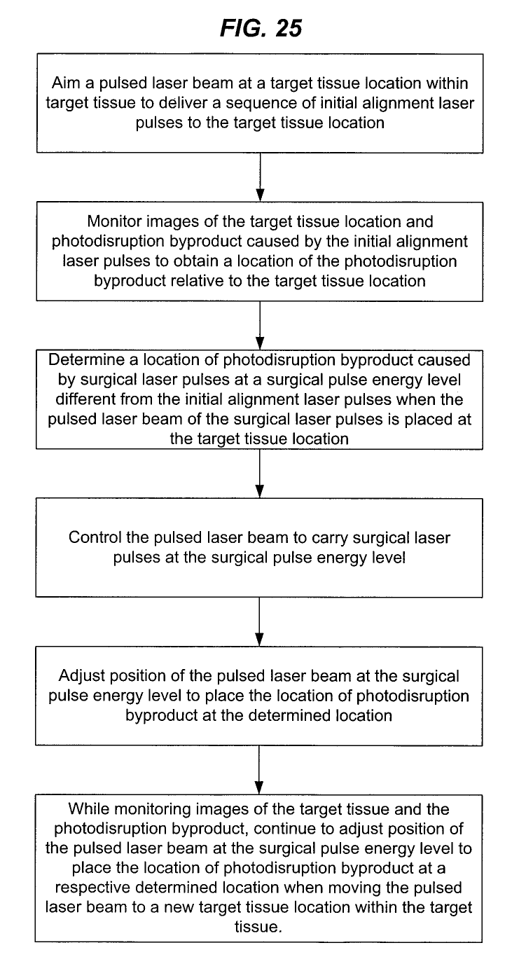

FIG. 25 shows one implementation of the laser alignment method based on imaging photodisruption byproduct in the target tissue in laser surgery. In this method, a pulsed laser beam is aimed at a target tissue location within target tissue to deliver a sequence of initial alignment laser pulses to the target tissue location. The images of the target tissue location and photodisruption byproduct caused by the initial alignment laser pulses are monitored to obtain a location of the photodisruption byproduct relative to the target tissue location. The location of photodisruption byproduct caused by surgical laser pulses at a surgical pulse energy level different from the initial alignment laser pulses is determined when the pulsed laser beam of the surgical laser pulses is placed at the target tissue location. The pulsed laser beam is controlled to carry surgical laser pulses at the surgical pulse energy level. The position of the pulsed laser beam is adjusted at the surgical pulse energy level to place the location of photodisruption byproduct at the determined location. While monitoring images of the target tissue and the photodisruption byproduct, the position of the pulsed laser beam at the surgical pulse energy level is adjusted to place the location of photodisruption byproduct at a respective determined location when moving the pulsed laser beam to a new target tissue location within the target tissue.

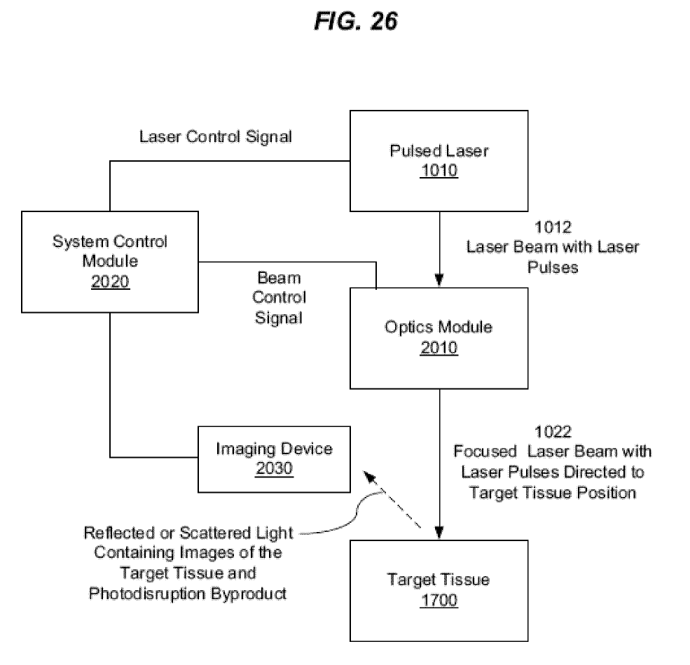

FIG. 26 shows an exemplary laser surgical system based on the laser alignment using the image of the photodisruption byproduct. An optics module 2010 is provided to focus and direct the laser beam to the target tissue 1700. The optics module 2010 can include one or more lenses and may further include one or more reflectors. A control actuator is included in the optics module 2010 to adjust the focusing and the beam direction in response to a beam control signal. A system control module 2020 is provided to control both the pulsed laser 1010 via a laser control signal and the optics module 2010 via the beam control signal. The system control module 2020 processes image data from the imaging device 2030 that includes the position offset information for the photodisruption byproduct 1702 from the target tissue position in the target tissue 1700. Based on the information obtained from the image, the beam control signal is generated to control the optics module 2010 which adjusts the laser beam. A digital processing unit is included in the system control module 2020 to perform various data processing for the laser alignment.

The imaging device 2030 can be implemented in various forms, including an optical coherent tomography (OCT) device. In addition, an ultrasound imaging device can also be used. The position of the laser focus is moved so as to place it grossly located at the target at the resolution of the imaging device. The error in the referencing of the laser focus to the target and possible non-linear optical effects such as self focusing that make it difficult to accurately predict the location of the laser focus and subsequent photodisruption event. Various calibration methods, including the use of a model system or software program to predict focusing of the laser inside a material can be used to get a coarse targeting of the laser within the imaged tissue. The imaging of the target can be performed both before and after the photodisruption. The position of the photodisruption by products relative to the target is used to shift the focal point of the laser to better localize the laser focus and photodisruption process at or relative to the target. Thus the actual photodisruption event is used to provide a precise targeting for the placement of subsequent surgical pulses.

Photodisruption for targeting during the diagnostic mode can be performed at a lower, higher or the same energy level that is required for the later surgical processing in the surgical mode of the system. A calibration may be used to correlate the localization of the photodisruptive event performed at a different energy in diagnostic mode with the predicted localization at the surgical energy because the optical pulse energy level can affect the exact location of the photodisruptive event. Once this initial localization and alignment is performed, a volume or pattern of laser pulses (or a single pulse) can be delivered relative to this positioning. Additional sampling images can be made during the course of delivering the additional laser pulses to ensure proper localization of the laser (the sampling images may be obtained with use of lower, higher or the same energy pulses). In one implementation, an ultrasound device is used to detect the cavitation bubble or shock wave or other photodisruption byproduct. The localization of this can then be correlated with imaging of the target, obtained via ultrasound or other modality. In another embodiment, the imaging device is simply a biomicroscope or other optical visualization of the photodisruption event by the operator, such as optical coherence tomography. With the initial observation, the laser focus is moved to the desired target position, after which a pattern or volume of pulses is delivered relative to this initial position.

As a specific example, a laser system for precise subsurface photodisruption can include means for generating laser pulses capable of generating photodisruption at repetition rates of 100-1000 Million pulses per second, means for coarsely focusing laser pulses to a target below a surface using an image of the target and a calibration of the laser focus to that image without creating a surgical effect, means for detecting or visualizing below a surface to provide an image or visualization of a target the adjacent space or material around the target and the byproducts of at least one photodisruptive event coarsely localized near the target, means for correlating the position of the byproducts of photodisruption with that of the sub surface target at least once and moving the focus of the laser pulse to position the byproducts of photodisruption at the sub surface target or at a relative position relative to the target, means for delivering a subsequent train of at least one additional laser pulse in pattern relative to the position indicated by the above fine correlation of the byproducts of photodisruption with that of the sub surface target, and means for continuing to monitor the photodisruptive events during placement of the subsequent train of pulses to further fine tune the position of the subsequent laser pulses relative to the same or revised target being imaged.

The above techniques and systems can be used deliver high repetition rate laser pulses to subsurface targets with a precision required for contiguous pulse placement, as needed for cutting or volume disruption applications. This can be accomplished with or without the use of a reference source on the surface of the target and can take into account movement of the target following applanation or during placement of laser pulses.

While this specification contains many specifics, these should not be construed as limitations on the scope of any invention or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

1. A method for integrated eye surgery, comprising the steps of:
determining a cataract-target region in a lens of the eye;
applying cataract-laser pulses to photodisrupt a portion of the determined cataract-target region;
determining a glaucoma-target region in a peripheral region of the eye; and
applying glaucoma-laser pulses to create one or more incisions in the glaucoma-target region by photodisruption; wherein
the steps of the method are performed within an integrated surgical procedure.

2. The method of claim 1, wherein:
the applying the cataract-laser pulses step is performed before the applying the glaucoma-laser pulses step.

3. The method of claim 1, wherein:
the applying the cataract-laser pulses step is performed after the applying the glaucoma-laser pulses step.

4. The method of claim 1, wherein:
the applying the cataract-laser pulses step is performed at least partially concurrently with the applying the glaucoma-laser pulses step.

5. The method of claim 1, the applying glaucoma-laser pulses step comprising:
applying the glaucoma-laser pulses into at least one of a sclera, a limbal region, an ocular angle portion, or an iris root.

6. The method of claim 1, the applying glaucoma-laser pulses step comprising:
applying the glaucoma-laser pulses according to a pattern in relation to at least one of a trabeculoplasty, iridotomy or iridectomy.

7. The method of claim 1, the applying glaucoma-laser pulses step comprising:
applying the glaucoma-laser pulses to form at least one of a drain channel or a humor outflow opening.

8. The method of claim 7, the method comprising:
inserting an implantable device into one of the drain channel or the humor outflow opening.

9. The method of claim 7, wherein:
the drain channel or the humor outflow opening is configured to connect an anterior chamber of a surgical eye to a surface of the surgical eye, thereby allowing a reduction of an intraocular pressure of an aqueous humor in the surgical eye.

10. The method of claim 7, comprising:
utilizing one laser for applying both the cataract-laser pulses and the glaucoma-laser pulses.

11. The method of claim 10, the applying glaucoma-laser pulses step comprising:
applying the glaucoma-laser pulses to an optimized glaucoma-target region,
wherein a location of the optimized glaucoma-target region is selected
to scatter the glaucoma-laser pulses less than a sclera of the eye, and
to perturb an optical pathway of the eye by the formed drain channel less than a centrally formed drain channel.

12. The method of claim 1, wherein:
the glaucoma-target region is one of
a limbus-sclera boundary region or a limbus-cornea intersection region.

13. The method of claim 1, the applying glaucoma-laser pulses step comprising:
applying the glaucoma-laser pulses to form a drain channel in a direction selected to optimize the competing requirements of
scattering the glaucoma-laser pulses less than a sclera of the eye, and
perturbing an optical pathway eye less than a centrally formed drain channel.

14. The method of claim 1, comprising:
determining a placement of the cataract-laser pulses and a placement of the glaucoma-laser pulses in a coordinated manner.

15. The method of claim 14, comprising:
imaging a photodisruption achieved by the cataract-laser pulses; and
determining at least portions of the glaucoma-target region in response to the imaged photodisruption.

16. The method of claim 14, comprising:
imaging a photodisruption achieved by the glaucoma-laser pulses; and
determining at least portions of the cataract-target region in response to the imaged photodisruption.

17. The method of claim 1, wherein:
the cataract-laser pulses are applied with a cataract-laser wavelength λ-c; and
the glaucoma-laser pulses are applied with a glaucoma-laser wavelength λ-g.

18. The method of claim 1, wherein:
the cataract-laser pulses are applied through a cataract-patient interface; and
the glaucoma-laser pulses are applied through a glaucoma-patient interface.

19. A multi-purpose ophthalmic surgical system, comprising:
a multi-purpose laser, configured
to place cataract-laser pulses into a cataract-target region, and
to place glaucoma-laser pulses into a glaucoma-target region; and
an imaging system, configured to image a photodisruption caused by at least one of the cataract-laser pulses and the glaucoma-laser pulses.

20. The multi-purpose ophthalmic surgical system of claim 19, wherein:
the multi-purpose laser is configured
to apply the cataract-laser pulses with a cataract-laser wavelength λ-c, and
to apply the glaucoma-laser pulses with a glaucoma-laser wavelength of λ-g.

21. The multi-purpose ophthalmic surgical system of claim 19, wherein:
the multi-purpose laser is configured
to apply the cataract-laser pulses through a cataract-patient interface, and
to apply the glaucoma-laser pulses through a glaucoma-patient interface.

22. The multi-purpose ophthalmic surgical system of claim 19, wherein:
the cataract-laser pulses and the glaucoma-laser pulses are applied by the same laser.

23. A method for integrated eye surgery, comprising the steps of:
determining a cataract-target region in a lens of the eye;
applying cataract-laser pulses to photodisrupt a portion of the determined cataract-target region;
determining an astigmatism-target region in a central, mid, or peripheral region of the eye; and
applying astigmatism correcting-laser pulses to create one or more incisions in the astigmatism-target region by photodisruption; wherein
the steps of the method are performed within an integrated surgical procedure.

24. The method of claim 23, comprising:
imaging a photodisruption achieved by the cataract-laser pulses; and
determining at least portions of the astigmatism-target region in response to the imaged photodisruption.

25. A multi-purpose ophthalmic surgical system, comprising:
a multi-purpose laser, configured
to place cataract-laser pulses into a cataract-target region, and
to place astigmatism-laser pulses into an astigmatism-target region; and
an imaging system, configured to image a photodisruption caused by at least one of the cataract-laser pulses and the astigmatism-laser pulses.
